SEQUENCE LISTING (12 sequences)

SEQ ID NO: 1 - DNA, 21 bp, Artificial Sequence
  Synthetic polynucleotide
tgtccccactgccaacgtgtc

SEQ ID NO: 2 - DNA, 24 bp, Artificial Sequence
  Synthetic polynucleotide
agcgtcaaaggtggaggagtgggt

SEQ ID NO: 3 - DNA, 20 bp, Artificial Sequence
  Synthetic polynucleotide
aactaatgccaccaccaagg

SEQ ID NO: 4 - DNA, 20 bp, Artificial Sequence
  Synthetic polynucleotide
aagacgtttcttggcctgag

SEQ ID NO: 5 - DNA, 20 bp, Artificial Sequence
  Synthetic polynucleotide
tgcgagtacaatgggacaac

SEQ ID NO: 6 - DNA, 20 bp, Artificial Sequence
  Synthetic polynucleotide
gttgccgattctgaaagagc

SEQ ID NO: 7 - DNA, 22 bp, Artificial Sequence
  Synthetic polynucleotide
gtgtcagtgaatgcttctgtcc

SEQ ID NO: 8 - DNA, 20 bp, Artificial Sequence
  Synthetic polynucleotide
gagttgctgaatgtgcatcg

SEQ ID NO: 9 - DNA, 20 bp, Artificial Sequence
  Synthetic polynucleotide
tgagaacaacacggcctatg

SEQ ID NO: 10 - DNA, 20 bp, Artificial Sequence
  Synthetic polynucleotide
tcaaagcctgccacaaagag

SEQ ID NO: 11 - DNA, 20 bp, Artificial Sequence
  Synthetic polynucleotide
caacgtggaaaaggcgttac

SEQ ID NO: 12 - DNA, 20 bp, Artificial Sequence
  Synthetic polynucleotide
tccccttcagaaacacaacc

(19) United States
(12) Patent Application Publication
(10) Pub. No.: US 2017/0209621 A1
(43) Pub. Date: Jul. 27, 2017

(54) USE OF CHORDIN-LIKE 1 OR COLLAGEN VI WITH MESENCHYMAL STEM CELLS

(71) Applicant: The Board of Trustees of the Leland Stanford Junior University, Palo Alto, CA (US)

(72) Inventors: Nidhi Bhutani, Stanford, CA (US); Piera Smeriglio, Stanford, CA (US); William J. Maloney, Stanford, CA (US); Sarah E. B. Taylor, Stanford, CA (US)

(21) Appl. No.: 15/411,367

(22) Filed: Jan. 20, 2017

Related U.S. Application Data
(60) Provisional application No. 62/281,331, filed on Jan. 21, 2016.

(51) Int. Cl. A61L 27/38; A61L 27/36; A61L 27/22; C12N 5/0775; A61L 27/24

(52) U.S. Cl. CPC A61L 27/3834; C12N 5/0663; A61L 27/24; A61L 27/227; A61L 27/3687; A61L 27/3852; C12N 2501/998; A61L 2400/06; A61L 2430/06

(57) ABSTRACT

There are provided inter alia reagents for increasing growth, expansion or survival of mesenchymal stem cells in cell culture, and methods of use thereof.

### CROSS-REFERENCE TO RELATED APPLICATIONS

This application claims the priority and benefit of U.S. Provisional Patent Application 62/281,331, filed Jan. 21, 2016, which is incorporated herein by reference in its entirety and for all purposes.

### INCORPORATION BY REFERENCE OF SEQUENCE LISTING

The contents of the text file named 41243-516001US_ST25.TXT, which was created on Jan. 20, 2017, and is 2,768 bytes in size, are hereby incorporated by reference in their entireties.

### BACKGROUND

Articular cartilage has a poor inherent capacity for regeneration. Although tissue regeneration generally declines with age, cartilage injuries are problematic even in young adults and can often lead to post-traumatic osteoarthritis. Cartilage tissue engineering is therefore a relevant and attractive strategy for cartilage injuries. Current cell-based approaches for engineering cartilage have, however, had limited success. Cell sources commonly utilized for engineering cartilage include autologous chondrocytes, mesenchymal stem cells (MSC) or adipose-derived stem cells (ADSC). The major challenges common to all these cell sources are the paucity of available cells and the generation of functionally inferior fibrocartilage rather than articular cartilage. In addition, in vitro expansion of autologous chondrocytes can lead to dedifferentiation and loss of chondrogenic phenotype. Expansion of MSC and ADSC in vitro is also challenging as it leads to a rapid loss of stem cell properties.

Moreover, damage to articular cartilage is a common injury and can frequently lead to degenerative disorders (e.g., osteoarthritis) with aging. Due to its low cellularity and highly avascular nature, articular cartilage has a limited ability to heal, and consequently cartilage injuries frequently require surgical intervention or lead to osteoarthritis (OA). Currently employed regenerative treatments for cartilage defects involve microfracture of the subchondral bone to release bone marrow derived mesenchymal stem cells (MSC) into the cartilage or cell-based techniques including autologous chondrocyte implantation (ACT). Both the techniques are limited by donor availability, the need for multiple surgeries, and limited cell numbers.

Thus, there is a need in the art to identify soluble factors that can enhance the growth and expansion of mesenchymal stem cell populations while maintaining their phenotype and function. Solutions to these and other problems in the art are provided herein.

### SUMMARY

In a first aspect, there is provided an in vitro mesenchymal stem cell culture including a plurality of mesenchymal stem cells and an amount of a protein selected from chordin-like protein 1 (CHRDL1) and collagen VI effective to increase in vitro growth, expansion or survival of the plurality of mesenchymal stem cells relative to the absence of the protein.

In another aspect, there is provided a method of increasing growth, expansion or survival of a plurality of mesenchymal stem cells. The method includes contacting the plurality of mesenchymal stem cells with an effective amount of a protein selected from chordin-like protein 1 (CHRDL1) and collagen VI, thereby increasing growth, expansion or survival of a plurality of mesenchymal stem cells.

In another aspect, there is provided a method of administering a plurality of mesenchymal stem cells to a subject in need thereof. The method includes contacting in vitro the plurality of mesenchymal stem cells with an effective amount of a protein selected from chordin-like protein 1 (CHRDL1) and collagen VI thereby increasing growth, expansion or survival of the plurality of mesenchymal stem cells to form a treated plurality of mesenchymal stem cells. The method further includes administering the treated plurality of mesenchymal stem cells to the subject.

In another aspect, there is provided a method of increasing cartilage growth in a subject in need thereof. The method includes administering to the subject an effective amount of a protein selected from chordin-like protein 1 (CHRDL1) and collagen VI.

### BRIEF DESCRIPTION OF THE DRAWINGS

FIGS. 1A-1C. Juvenile chondrocytes display unique gene expression profiles. FIG. 1A: Schematic representing the unique set of as yet unknown genes expressed by juvenile chondrocytes (left). FIG. 1B: The top 20 genes identified in juvenile chondrocytes with a 1.5× or greater increase in expression when compared to adult and hMSCs and their relative fold change. A star marks genes that were identified for further analyses. FIG. 1C: The top 10 pathways identified by network analysis of the 560 genes that are upregulated by 1.5-fold or greater in the juvenile chondrocytes when compared to the adult chondrocytes.

FIGS. 2A-2C. Common ‘stemness’ genes upregulated in both juvenile chondrocytes and hMSCs when compared to adult chondrocytes. FIG. 2A: Schematic representing the overlapping set of genes expressed by juvenile chondrocytes (left) and hMSCs (right). The number of genes with a 1.5-fold or greater increase in expression when compared to adult chondrocytes is shown. FIG. 2B: The top 20 genes identified in both hMSCs and juvenile chondrocytes with a 1.5-fold or greater increase in expression when compared to adult chondrocytes and their relative fold change. FIG. 2C: The top 10 pathways, identified by network analysis of the ‘stemness’ genes that are upregulated by 1.5-fold or greater in both hMSCs and juvenile chondrocytes when compared to adult chondrocytes.

FIGS. 3A-3C. qPCR validation of gene expression levels of juvenile chondrocyte markers selected for further analysis in a wider-range of samples. FIG. 3A: Chordin-like 1 (CHRDL1); FIG. 3B: osteomodulin (OMD); FIG. 3C: Microfibrillar-associated protein 4 (MFAP4). X-axis indicates sample age. hMSC=human mesenchymal stem cells: Juvenile—6 mo=6 months, 18 mo=18 months, 6 yrs=6-year old; Adult—18M=18-year old male, 27F=27 year old female, 34M=34 year old male; OA (osteoarthritic samples): 64F=64 year old female, 79F=79 year old female. Statistics were performed using a two-tailed Student's t-test comparing each sample to the 34M sample. * p<0.05.

FIGS. 4A-4B. Protein expression of selected juvenile chondrocyte specific markers. FIG. 4A: Immunostaining of juvenile (6 mo=6 months, 6 yrs=6 years) and adult chondrocytes (34 yrs 34 years) with an antibody specific for chordin-like 1 (CHRDL1). Nuclei are counterstained with DAPI. Scale bar=30 μm. FIG. 4B: Immunostaining of juvenile (6 mo=6 months, 6 yrs=6-year old) and adult chondrocytes (34M=34 year old male) with an antibody specific for microfibrillar-associated protein 4 (MFAP4). Nuclei are counterstained with DAPI. Scale bar=30 μm.

FIGS. 5A-5B. Chordin-like 1 stimulates proliferation of hMSCs but MEAP4 does not.

FIG. 5A: hMSCs and adult chondrocyte growth upon treatment with chordin-like 1 (CHRDL1, 100 μM) in monolayer cultures for 4 days. CHRDL1 was replenished daily. hMSC (19F)=hMSC from 19 year old female donor; hMSC (30M)=hMSC from 30 year old male donor; Adult chondrocyte (34M)=adult chondrocytes from 34 year old male donor. FIG. 5B: hMSCs and adult chondrocyte growth upon treatment with microfibrillar-associated protein 4 (MFAP4, 25 μM) in monolayer cultures for 4 days. MFAP4 was replenished daily. (hMSC (19F)=hMSC from 19 year old female donor; hMSC (30M)=hMSC from 30 year old male donor; Adult chondrocyte (34M)=adult chondrocytes from 34 year old male donor. Fold increase in cell number is indicated relative to Day 0 and normalized to the control at each time point. Donor age is indicated in parentheses. Data represent three biological replicates and are expressed as mean±S.D. (standard deviation). * denotes statistical significant, where <0.05. ̂ denotes approaching significant, p<0.09.

FIGS. 6A-6B. Juvenile factors Collagen. VI (Col VI) and Chordin-like 1 (CHRDL1) stimulate proliferation of hMSC. hMSC were treated with Col V1 (100 nM) (FIG. 6A) and chordin-like 1 (CHRDL1, 100 nM) (FIG. 6B) in monolayer cultures for 4 days. Col V1 and CHRDL1 were replenished daily. Fold increase in cell number is indicated relative to Day 0 and normalized to the conrol a each time point.

FIGS. 7A-7E. FIGS. 7A-7D depict histograms of gene expression levels relative to control for chondrocytes markers SOX9, COL2A, and ACAN, and mesenchymal stem cell marker NKX2.5, respectively, assayed in hMSC and adult human chondrocytes (34M) after treatment with CHRDL1 or MFPA4 for 2 days. FIG. 7E: FACS analysis for hMSC ‘stemness’ markers, CD90 (Thy-1) and CD105 (Endoglin) in hMSC treated with CHRDL1 for 2 days.

FIGS. 8A-8B. Col VI treatment enhances hMSC proliferation. hMSC from 19 years old male donor (FIG. 8A) and 30 years old male donor (FIG. 8B) upon treatment with soluble Col VI (2.5 μg/ml) in monolayer cultures for 4 days. Col VI was replenished daily. Fold increase in cell number is indicated relative to day 0 and normalized to the control at each time point. Data represent three biological replicates and are expressed as mean±SD. *Statistical significance where p<0.05.

FIGS. 9A-9C. Col VI treatment maintains mesenchymal Stem Cell phenotype. FIG. 9A: FACS analysis for hMSC stemness markers, CD90 (Thy-1) and CD105 (Endoglin) expression in hMSC treated with control or 2.5 μg/ml. Col VI for 2 days. FIG. 9B: FACS analyses of chondrogenic markers SOX9 and CD44 expression in hMSC treated with control or 2.5 μg/ml Col VI for 2 days. FIG. 9C: Gene expression for chondrocyte markers Acan, Sox9 and Col2a1 was assayed in hMSC after treatment with control or 2.5 μg/ml Col VI for 2 days.

FIGS. 10A-10B. Col VI treatment retains the chondrogenic differentiation potential of hMSC. FIG. 10A: Glycosaminoglycan (GAG) content, DNA content and GAG normalized to DNA content in pellets seeded with control and COLVI-treated hMSC after 21 days of culture. FIG. 10B: Gene expression levels for chondrocyte markers Acan, Sox9 and MMP13 were assayed in hMSC pre-treated with COLVI and cultured for 21 days.

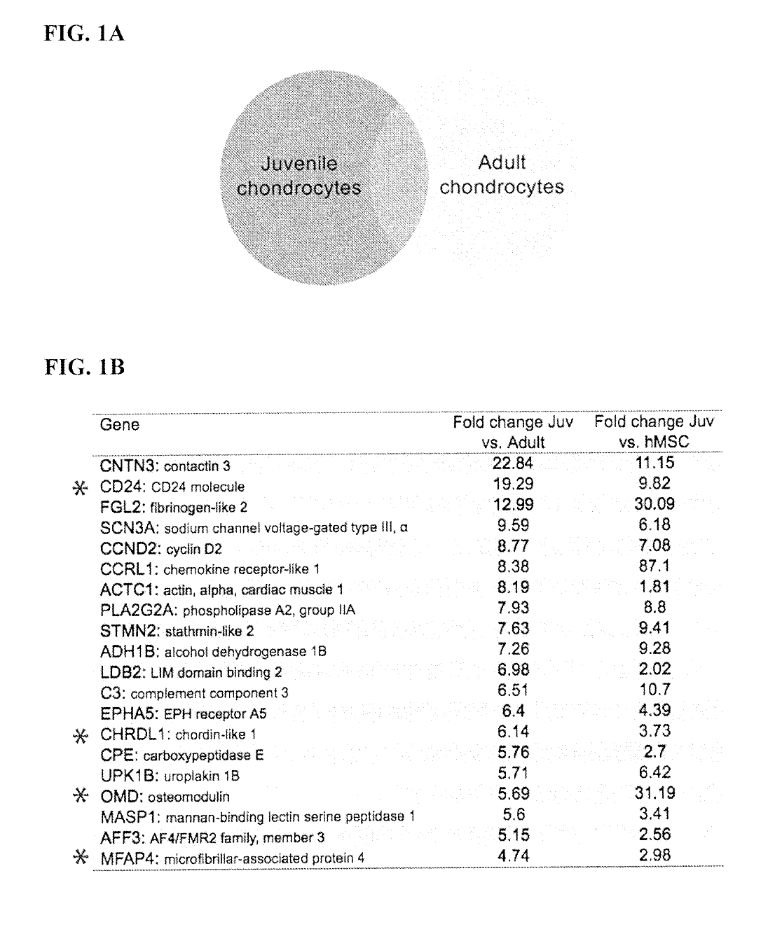

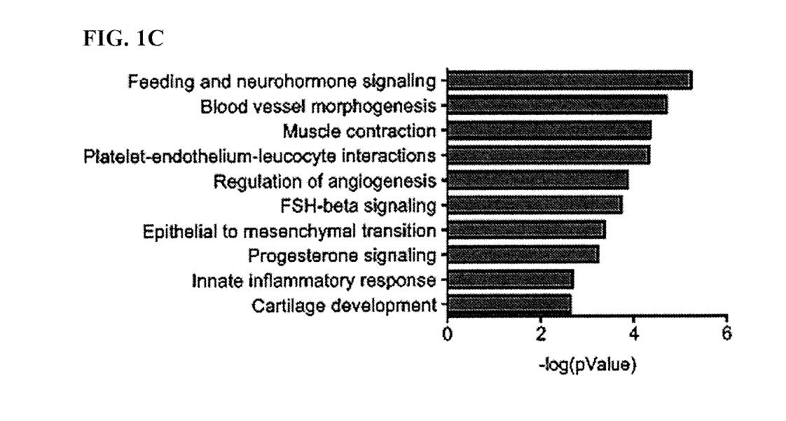

FIGS. 1.1A-11C. Col VI-treated hMSC show efficient cartilage differentiation similar to control hMSC. Chondrogenic marker expression in cartilage pellets after 21 days of culture for control or Col VI-treated hMSC. Immunofluorescence staining for (FIG. 11A) collagen 2a1 (COL2A1), (FIG. 11B) collagen 6a1 (COL6A1) and (FIG. 11C) aggrecan (AGC). Cell nuclei were stained with DAPI (4′,6-diamidino-2-phenylindole).

### DETAILED DESCRIPTION

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., T. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The abbreviations used herein have their conventional meaning within the chemical and biological arts. The chemical structures and formulae set forth herein are constructed according to the standard rules of chemical valency known in the chemical arts.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

The term “mesenchymal stem cell,” “MSC” and the like refer, in the usual and customary sense, to multipotent stromal cells that can differentiate into a variety of cell types, including osteoblasts, chondrocytes, myocytes, adipocytes, and the like. The term “multipotent” refers, in the usual and customary sense, to a progenitor cells having the potential to differentiate into any of a plurality of cell types. The term “stromal cell” refers, in the usual and customary sense, to connective tissue cells found, e.g., in uterine mucosa, prostate, bone marrow, and the ovary, and which function, e.g., to support the function of the parenchymal cells. In embodiments, MSC cells are capable of replication as undifferentiated cells. In embodiments, MSC cells are capable of differentiating into bone, cartilage, fat, muscle, tendon and marrow stroma. In embodiments, MSC cells are normal human bone marrow derived Mesenchymal Stem Cells (hMSC). Bone marrow derived Mesenchymal Stem Cells are mesenchymal stem cells isolated from human bone marrow. In embodiments, MSC cells are hMSC cells that are isolated from normal (non-diabetic) adult human bone marrow withdrawn from bilateral punctures of the posterior iliac crests of normal volunteers. In embodiments, normal hMSCs have been cryopreserved after the second passage and are suggested to be used by no later than the fifth passage.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

The terms “chordin-like protein 1, “CHRDL1” and the like refer, in the usual and customary sense, to any of the recombinant or naturally-occurring forms of chordin-like protein 1 or variants or homologs thereof that maintain chordin-like protein 1 activity (e.g., within at least 50%, 80%, 90%, 95%, 96%, 97%, 98%, 99% or 100% activity compared to CHRDL1). In embodiments, the variants or homologs have at least 80%, 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CHRDL1 polypeptide. In embodiments, the variants or homologs have the full length of a naturally occurring CHRDL1 polypeptide. In embodiments, the variants or homologs have functional fragments of a naturally occurring CHRDL1 polypeptide. In embodiments, the variants or homologs are about 1-458 amino acids in length. In embodiments, the variants or homologs are about 1-400 amino acids in length. In embodiments, the variants or homologs are about 1-350 amino acids in length. In embodiments, the variants or homologs are about 1-300 amino acids in length. In embodiments, the variants or homologs are about 1-250 amino acids in length. In embodiments, the variants or homologs are about 1-200 amino acids in length. In embodiments, the variants or homologs are about 1-150 amino acids in length. In embodiments, the variants or homologs are about 1-100 amino acids in length. In embodiments, the variants or homologs are about 1-50 amino acids in length. In embodiments, the variants or homologs do not have signal peptide. In embodiments, CHRDL1 is the protein as identified by the NCBI sequence reference NP 001137453.1, NP 001137454.1, NP 001137455.2, NP 660277.2, or homolog (e.g., having at least 80%, 85%, 86%, 87%, 88%, 89%, 90%, 91%, 92%, 93%, 94%, 95%, 96%, 97%, 98%, 99% or 100% amino acid sequence identity) or functional fragment thereof. In embodiments, CHRDL1 is the protein as identified by the NCBI sequence reference NP_001137453.1 or homolog (e.g., having at least 80%, 85%, 86%, 87%, 88%, 89%, 90%, 91%, 92%, 93%, 94%, 95%, 96%, 97%, 98%, 99% or 100% amino acid sequence identity) or functional fragment thereof).

The terms “collagen VI,” “Col VI” and the like refer, in the usual and customary sense, to a form of collagen primarily associated with the extracellular matrix of skeletal muscle. The terms also refer to any of the recombinant or naturally-occurring forms of collagen VI or variants or homologs thereof that maintain collagen VI activity (e.g., within at least 50%, 80%, 90%, 95%, 96%, 97%, 98%, 99% or 100% activity compared to collagen VI). In embodiments, the variants or homologs have at least 80%, 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring collagen VI polypeptide. In embodiments, the variants or homologs have the full length of a naturally occurring collagen VI polypeptide. In embodiments, the variants or homologs have functional fragments of a naturally occurring collagen VI polypeptide. In embodiments, the variants or homologs are about 1-1028 amino acids in length. In embodiments, the variants or homologs are about 1-1000 amino acids in length. In embodiments, the variants or homologs are about 1-950 amino acids in length. In embodiments, the variants or homologs are about 1-900 amino acids in length. In embodiments, the variants or homologs are about 1-850 amino acids in length. In embodiments, the variants or homologs are about 1-800 amino acids in length. In embodiments, the variants or homologs are about 1-750 amino acids in length. In embodiments, the variants or homologs are about 1-700 amino acids in length. In embodiments, the variants or homologs are about 1-650 amino acids in length. In embodiments, the variants or homologs are about 1-600 amino acids in length. In embodiments, the variants or homologs are about 1-650 amino acids in length. In embodiments, the variants or homologs are about 1-600 amino acids in length. In embodiments, the variants or homologs are about 1-550 amino acids in length. In embodiments, the variants or homologs are about 1-500 amino acids in length. In embodiments, the variants or homologs are about 1-450 amino acids in length. In embodiments, the variants or homologs are about 1-400 amino acids in length. In embodiments, the variants or homologs are about 1-350 amino acids in length. In embodiments, the variants or homologs are about 1-300 amino acids in length. In embodiments, the variants or homologs are about 1-250 amino acids in length. In embodiments, the variants or homologs do not have signal peptide. In embodiments, Col IV is the protein as identified by the NCBI sequence reference NP_001839, NP_001840, NP_478054, NP_478055, NP_004360, NP_476505, NP_476506, NP_476507, NP_476508, NP_001265227, NP_694996 or homolog (e.g., having at least 80%, 85%, 86%, 87%, 88%, 89%, 90%, 91%, 92%, 93%, 94%, 95%, 96%, 97%, 98%, 99% or 100% amino acid sequence identity) or functional fragment thereof. In embodiments, Col IV is the protein mixture of one or more molecules as identified by the NCBI sequence reference NP_001839, NP_001840, NP_478054, NP_478055, NP_004360, NP_476505, NP_476506, NP_476507, NP_476508, NP_001265227, NP_694996 or homolog (e.g., having at least 80%, 85%, 86%, 87%, 88%, 89%, 90%, 91%, 92%, 93%, 94%, 95%, 96%, 97%, 98%, 99% or 100% amino acid sequence identity) or functional fragment thereof. In embodiments, Col IV is the protein mixture of molecules as identified by the NCBI sequence reference NP_001839, NP_001840, NP_478054, NP_478055, NP_004360, NP_476505, NP_476506. NP_476507, NP_476508, or homolog (e.g., having at least 80%, 85%, 86%, 87%, 88%, 89%, 90%, 91%, 92%, 93%, 94%, 95%, 96%, 97%, 98%, 99% or 100% amino acid sequence identity) or functional fragment thereof. In embodiments, collagen VI molecules are made up of three alpha chains: α1(VI), α2(VI), and α3(VI) and are associated with the genes COL6A1, COL6A2, COL6A3 and COL6A5. In embodiments, collagen VI used herein is a mixture of protein products encoded by genes COL6A1, COL6A2, and COL6A3.

The term “growth” in the context of cell cultures disclosed herein refers, in the usual and customary sense, to viability including proliferation of the cell culture. The term “expansion” in this context refers, in the usual and customary sense, to growth of the cell culture with respect to number of cells and/or size of cells. The term “survival” in this context refers, in the usual and customary sense, to continuing viability of the culture.

The terms “subject,” “individual,” and “patient” are used interchangeably herein to refer to a vertebrate, preferably a mammal, more preferably a human. The mammal can be e.g., a human or appropriate non-human mammal, such as primate, mouse, rat, dog, cat, cow, horse, goat, camel, sheep or a pig. The subject can also be a bird or fowl. Tissues, cells and their progeny of a biological entity obtained in vivo or cultured in vitro are also encompassed.

In embodiments, a “subject in need thereof” or “a patient” may be a subject having a cartilage-related disease. The term “cartilage-related disease” refers to a structural and/or biological imperfection in cartilage tissue such as but not limited to a break, tear, void or other disintegration of the tissue, which is caused by a disease, injury or condition and which can benefit from cartilage repair, replacement, or augmentation, such as, in non-limiting example, athletic injury, traumatic injury, congenital disorders, osteoarthritis and/or pathologic joint degeneration. In some embodiments, non-limiting examples of phenotypic indicators of cartilage-related disease include proteoglycan loss, joint space narrowing, collagen degradation, and destruction of cartilage. In embodiments, a cartilage-related disease also encompasses cartilage degeneration due to aging. In embodiments, cartilage-related disease refers to one or more of post-traumatic osteoarthritis, rheumatoid arthritis, chondromatosis, costochondritis, relapsing polychondritis, herniation, chondrolysis, achondroplasia, chondrodysplasia, chondroma, and chondrosarcoma.

In embodiments, a “subject in need thereof” may be a subject having cartilage damage. Cartilage is a connective tissue found in many parts of the body. Although it is a tough and flexible material, it is relatively easy to damage. People with cartilage damage commonly experience joint pain, stiffness, and inflammation (swelling). Cartilage can become damaged as a result of a sudden injury, such as a sports injury, or gradual wear and tear (osteoarthritis). In embodiments, cartilage damage is a minor cartilage injury. In embodiments, cartilage damage is severe cartilage damage that may require surgery or even transplantation.

As used herein, a “injury” is any disruption, from whatever cause, of normal anatomy (internal and/or external anatomy) including but not limited to traumatic injuries such as mechanical (i.e. contusion, penetrating, crush), thermal, chemical, electrical, radiation, concussive and incisional injuries; elective injuries such as operative surgery and resultant incisional hernias, fistulas, etc.; acute injuries, chronic injuries, infected injuries, and sterile injuries.

In embodiments, a “subject in need thereof” is a subject in need of a transplantation procedure. In embodiments, a “subject in need thereof” is a subject in need of cartilage growth (such as cartilage repair).

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, electrocardiogram, echocardiography, radio-imaging, nuclear scan, and/or stress testing, neuropsychiatric exams, and/or a psychiatric evaluation.

An “effective amount” is an amount sufficient to accomplish a stated purpose (e.g. achieve the effect for which it is administered, treat a disease, reduce enzyme activity, increase enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art; Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch.

The cells and compositions described herein can be formulated to be compatible with its intended route of administration. Examples of routes of administration include parenteral, e.g., intravenous, intradermal, subcutaneous, oral (e.g., inhalation), transdermal (topical), and transmucosal administration.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The invention described herein is partially based upon the discovery that soluble CHRDL1 and Col VI can individually aid the proliferation and survival of human bone marrow-derived mesenchymal stem cells (MSC) and can therefore be useful as biologics to enhance stem cell function in cartilage repair. In embodiments, the identified soluble factors can be utilized as biologics (1) in cartilage repair procedures (e.g., repair of microfracture) to enhance endogenous stem cell mediated cartilage repair, (2) in combination with implantation of acellular scaffolds to enhance stem cell mediate cartilage repair, or (3) in cell-based tissue engineered products for cartilage repair based on the expansion and differentiation of mesenchymal stem cells.

In embodiments, the cell cultures and the methods described herein provide a simple yet efficient in vivo and in vitro method to expand MSCs while keeping their stem cell properties; and provide a simple yet efficient method to generate functional articular cartilage, thus overcoming the major challenges common in the field: lack of available MSC cells, the generation of functionally inferior fibrocartilage rather than articular cartilage, and a rapid loss of stem cell properties of cultured MSC cells.

### Compositions

In a first aspect, there is provided an in vitro mesenchymal stem cell culture which includes a plurality of mesenchymal stem cells and an amount of a protein selected from chordin-like protein 1 (CHRDL1) and collagen VI effective to increase in vitro growth, expansion or survival of said plurality of mesenchymal stem cells relative to the absence of said protein.

In embodiments, an effective amount is an amount that increases the proliferation/replication/survival of cultured mesenchymal stem cells by at least 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400% or more compared to the proliferation/replication/survival of cultured mesenchymal stem cells in the absence of the protein.

In embodiments, an effective amount of CHRDL1 is about 1-200 nM (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 nM). In embodiments, an effective amount of CHRDL1 is about 50-150 nMS. In embodiments, an effective amount of CHRDL1 is about 80-120 nM. In embodiments, an effective amount of CHRDL1 is about 100 nM.

In embodiments, an effective amount of Col VI is about 1-10 μg/ml (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9 or 10 μg/ml). In embodiments, an effective amount of Col VI is about 2.5-5 μg/ml (e.g., 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 μg/ml), In embodiments, an effective amount of Col VI is about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 μg/ml.

In embodiments, the protein is CHRDL1. In embodiments, the protein is purified CHRDL1. Purified proteins are at least about 60% by weight (dry weight) the compound of interest. In embodiments, the preparation is at least about 75%, more preferably at least about 90%, 91%, 92%, 93%, 94%, 95%, 96%, 97%, 98%, and most preferably at least about 99% or higher by weight the compound of interest. Purity can be measured by any appropriate standard method, for example, by High-performance liquid chromatography, polyacrylamide gel electrophoresis. In embodiments, the purified CHRDL1 is a mature CHRDL1. In embodiments, the purified CHRDL1 does not include a signal peptide.

In embodiments, the protein is recombinant CHRDL1. In embodiments, the recombinant CHRDL1 includes an expression tag (e.g., a His tag). In embodiments, the recombinant CHRDL1 does not include any expression tag. In embodiments, the recombinant CHRDL1 is a recombinant mature CHRDL1, In embodiments, the recombinant CHRDL1 does not include a signal peptide.

In embodiments, the protein is a CHRDL1 variant or a functional fragment thereof. In embodiments, the protein is a CHRDL1 variant having at least 80%, 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CHRDL1 polypeptide (e.g., CHRDL1 identified by the NCBI sequence reference NP_001137453.1, NP_001137454.1, NP_001137455.2, or NP_{—} 660277.2). In embodiments, the protein is a functional fragment (e.g., about 1-458, 1-400, 1-350, 1-300, 1-250, 1-200, 1-150, 1-100, or 1-50 amino acids in length) of CHRDL1.

In embodiments, the protein is collagen VI (Col VI). In embodiments, the protein is purified Col VI. In embodiments, the protein is recombinant Col VI. In embodiments, the protein is a Col VI variant or a functional fragment thereof. In embodiments, the protein is a Col VI variant having at least 80%, 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Col VI polypeptide (e.g., Col VI identified by the NCBI sequence reference NP_001839, NP_001840, NP_478054, NP_478055, NP_004360, NP_476505, NP_476506, NP_476507, NP_476508, NP_001265227 or NP_694996). In embodiments, the protein is a functional fragment (e.g., about 1-1028, 1-1000, 1-950, 1-900, 1-850, 1-800, 1-750, 1-700, 1-650, 1-600, 1-550, 1-500, 1-450, 1-400, 1-350, 1-300, 1-250, 1-200, 1-150, 1-100, or 1-50 amino acids in length) of Col VI.

In embodiments, the protein is CHRDL1 and Col VI described above.

In embodiments, the mesenchymal stem cell culture includes a liquid culture media. In embodiments, the mesenchymal stem cell culture is a liquid culture. In embodiments, the liquid culture media includes MSC growth supplement, L-glutamine, antibiotics (e.g., gentamicin, and amphotericin). In embodiments, the mesenchymal stem cell culture includes at least one insoluble component. In embodiments, the protein of the mesenchymal stem cell culture is soluble in the liquid culture media.

In embodiments, the mesenchymal stem cell culture does not include any growth factors or cytokines or compounds that are not necessary for basic growing/proliferation/survival of mesenchymal stem cells. In embodiments, the mesenchymal stem cell culture does not include h-insulin, indomethacin, IBMX (3-isobuty-1-methyl-xanthine), ascorbate, sodium pyruvate, proline, TGF-β3, β-glycerophosphate or any combination thereof.

In embodiments, the mesenchymal stem cells are isolated from one or more human subjects. In embodiments, the mesenchymal stem cells are isolated from the same subject who will be later administered with the cells. In embodiments, the mesenchymal stem cells are isolated from one or more subject who are not the subject being administered with the cells. In embodiments, the mesenchymal stem cells are isolated from a human subject with an age of about 15 to 60 (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60). In embodiments, the mesenchymal stem cells are isolated from a human subject with an age of about 18 to 30. In embodiments, the mesenchymal stem cells are isolated from a human subject with an age of about 30 to 60.

In embodiments, the mesenchymal stem cells are isolated from a female subject. In embodiments, the mesenchymal stem cells are isolated from a male subject.

In embodiments, the expanded mesenchymal stem cells from the in vitro mesenchymal stem cell culture express mesenchymal stem cell markers, such as CD105 and CD90. In embodiments, about at least 80%, 85%, 86%, 87%, 88%, 89%, 90%, 91%, 92%, 93%, 94%, 95%, 96%, 97%, 98%, 99% or 100% of the expanded mesenchymal stem cells express mesenchymal stem cell markers, such as CD105 and CD90.

In embodiments, the expanded mesenchymal stem cells from the in vitro mesenchymal stem cell culture have no alteration in the expression of the chondrogenic transcription factor Sox9 and/or the cartilage surface marker CD44. In embodiments, about at least 80%, 85%, 86%, 87%, 88%, 89%, 90%, 91%, 92%, 93%, 94%, 95%, 96%, 97%, 98%, 99% or 100% of the expanded mesenchymal stem cells have no alteration in the expression of Sox9 and/or CD44.

Expression levels may be determined by one of a number of known in vitro assay techniques, such as PCR based assays, in situ hybridization assays, flow cytometry assays, immunological or immunohistochemical assays.

In embodiments, the expanded mesenchymal stem cells from the in vitro mesenchymal stem cell culture maintain the differentiation ability to chondrocytes. In embodiments, about at least 80%, 85%, 86%, 87%, 88%, 89%, 90%, 91%, 92%, 93%, 94%, 95%, 96%, 97%, 98%, 99% or 100% of the expanded mesenchymal stem cells can be differentiated into chondrocytes under proper differentiation condition.

### Methods of Use

In another aspect, there is provided a method for increasing growth, expansion or survival of a plurality of mesenchymal stem cells. The method includes contacting the plurality of mesenchymal stem cells with an effective amount of a protein selected from chordin-like protein 1 (CHRDL1) and collagen VI thereby increasing growth, expansion or survival of a plurality of mesenchymal stem cells. In embodiments, the method is an in vitro method. In embodiments, the method is an in vivo method.

In embodiments, the method increases growth of a plurality of mesenchymal stem cells. In embodiments, the method increases expansion of a plurality of mesenchymal stem cells. In embodiments, the method increases survival of a plurality of mesenchymal stem cells. In embodiments, the method increases growth/expansion/survival of the mesenchymal stem cells by at least about 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400% or more compared to the growth/expansion/survival of mesenchymal stem cells in the absence of the protein.

In another aspect, there is provided a method for administering a plurality of mesenchymal stem cells to a subject in need thereof. In embodiments, the plurality of mesenchymal stem cells administered to the subject is the expanded mesenchymal stem cells from an in vitro culture described herein. In embodiments, the method includes contacting in vitro the plurality of mesenchymal stem cells with an effective amount of a protein selected from chordin-like protein 1 (CHRDL1) and collagen VI thereby increasing growth, expansion or survival of said plurality of mesenchymal stem cells to form a treated plurality of mesenchymal stem cells. The method further includes administering the treated plurality of mesenchymal stem cells to the subject.

Further to any method for administering a plurality of mesenchymal stem cells to a subject in need thereof, and embodiments thereof, in embodiments the method further includes administering a protein selected from chordin-like protein 1 (CHRDL1) and collagen VI.

In another aspect, there is provided a method for increasing cartilage growth in a subject in need thereof. In embodiments, the method includes administering to the subject an effective amount of expanded mesenchymal stem cells described herein. In embodiments, the method includes administering to the subject an effective amount of a protein selected from chordin-like protein 1 (CHRDL1) and collagen VI. In embodiments, the method further includes administering a plurality of mesenchymal stem cells.

Further to any method for increasing cartilage growth in a subject in need thereof, in embodiments, the administering is through injection. In embodiments, the injection is provided at or near a cartilage-deficient site.

In embodiments, the subject is in need of in any method described herein a transplantation procedure. In embodiments, the method further includes transplanting additional biological tissue to the subject. In embodiments, the subject is in need of in any method described herein requires cartilage growth. In embodiments, the cartilage growth is cartilage repair. In embodiments, the mesenchymal stem cells differentiate to cartilage cells after the administering in any method described herein. In embodiments, the subject in need thereof in any method described herein is a subject having a cartilage-related disease. The term “cartilage-related disease” refers to a structural and/or biological imperfection in cartilage tissue such as but not limited to a break, tear, void or other disintegration of the tissue, which is caused by a disease, injury or condition and which can benefit from cartilage repair, replacement, or augmentation, such as, in non-limiting example, athletic injury, traumatic injury, congenital disorders, osteoarthritis and/or pathologic joint degeneration

In embodiments, an effective amount of the protein used in any method described herein is an amount that increases the proliferation/replication/survival of mesenchymal stem cells by at least about 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400% or more compared to the proliferation/replication/survival of mesenchymal stem cells in the absence of the protein.

In embodiments, the protein used in any method described herein is CHRDL1. In embodiments, the protein used in any method described herein is purified CHRDL1. In embodiments, the protein used in any method described herein is recombinant CHRDL1.

In embodiments, the protein used in any method described herein is a CHRDL1 variant or a functional fragment thereof. In embodiments, the protein used in any method described herein is a CHRDL1 variant having at least 80%, 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CHRDL1 polypeptide (e.g., CHRDL1 identified by the NCBI sequence reference NP_001137453.1, NP_001137454.1, NP_001137455.2, NP_660277.2). In embodiments, the protein used in any method described herein is a CHRDL1 functional fragment (e.g., about 1-458, 1-400, 1-350, 1-300, 1-250, 1-200, 1-150, 1-100, or 1-50 amino acids in length).

In embodiments, an effective amount of CHRDL1 used in any method described herein is about 1-200 nM (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 nM). In embodiments, an effective amount of CHRDL1 used in any method described herein is about 50-150 nM. In embodiments, an effective amount of CHRDL1 used in any method described herein is about 80-120 nM. In embodiments, an effective amount of CIIRDL1 used in any method described herein is about 100 nM.

In embodiments, the protein used in any method described herein is collagen VI (Col VI). In embodiments, the protein used in any method described herein is purified Col. VI. In embodiments, the protein used in any method described herein is recombinant Col VI. In embodiments, the protein used in any method described herein is a Col VI variant or a functional fragment thereof. In embodiments, the protein used in any method described herein is a Col VI variant having at least 80%, 85%, 86%, 87%, 88%, 89%, 90%, 91%, 92%, 93%, 94%. 95%, 96%, 97%, 98%, 99% or 100% amino acid sequence identity across the whole sequence or a portion of the sequence (e.g. a 50, 100, 150 or 200 continuous amino acid portion) compared to a naturally occurring Col VI polypeptide (e.g., Col VI identified by the NCBI sequence reference NP_001839, NP_001840, NP_478054, NP_478055, NP_004360, NP_476505, NP_476506, NP_476507, NP_476508, NP_001265227 or NP_694996). In embodiments, the protein used in any method described herein is a functional fragment (e.g., about 1-1028, 1-1000, 1-950, 1-900, 1-850, 1-800, 1-750, 1-700, 1-650, 1-600, 1-550, 1-500, 1-450, 1-400, 1-350, 1-300, 1-250, 1-200, 1-150, 1-100, or 1-50 amino acids in length) of Col VI.

In embodiments, an effective amount of Col. VI used in any method described herein is about 1-10 μg/ml (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9 or 10 μg/ml). In embodiments, an effective amount of Col VI used in any method described herein is about 2.5-5 ug/ml (e.g., 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 μg/ml). In embodiments, an effective amount of Col VI used in any method described herein is about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 μg/ml.

In embodiments, the protein used in any method described herein is CHRDL1 and Col VI described above.

One of ordinary skill in the art may readily determine the appropriate concentration, or dose of the compositions disclosed herein for therapeutic administration. The ordinary artisan will recognize that a preferred dose is one that produces a therapeutic effect, such as preventing, treating and/or reducing inflammation associated with cartilage diseases, disorders and injuries, in a patient in need thereof. Of course, proper doses of the cells will require empirical determination at time of use based on several variables including but not limited to the severity and type of disease, injury, disorder or condition being treated; patient age, weight, sex, health; other medications and treatments being administered to the patient; and the like. An exemplary dose is in the range of about 0.25-2.0×10⁶ cultured MSC cells. Other dose ranges include 0.1-10.0×10⁶, 0.1-10.0×10⁷, 0.1-10.0×10⁸, 0.1-10.0×10⁹, 0.1-10.0×10¹⁰, 0.1-10×10¹¹, or 0.1-10.0×10¹² cells per dose or injection regimen.

An effective amount of cells may be administered in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of pharmaceutical composition. Where there is more than one administration of a pharmaceutical composition in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

Cells described herein or prepared from the methods described herein or protein described herein (CHRDL1 and/or Col VI) may be formulated for administration according to any of the methods disclosed herein in any conventional manner using one or more physiologically acceptable carriers optionally comprising excipients and auxiliaries. Proper formulation is dependent upon the route of administration chosen. The compositions may also be administered to the individual in one or more physiologically acceptable carriers. Carriers for cells may include, but are not limited to, solutions of normal saline, phosphate buffered saline (PBS), lactated. Ringer's solution containing a mixture of salts in physiologic concentrations, or cell culture medium.

In embodiments, the compositions described herein (e.g., cells described herein or prepared from the methods described herein or protein described herein (CHRDL1 and/or Col VI)) are administered by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, epidural, intracerebral, intraosseous, intracartilagenous, and intrasternal injection or infusion.

The timing of administration of the compositions described herein (e.g., cells described herein or prepared from the methods described herein or protein described herein (CHRDL1 and/or Col VI)) will depend upon the type and severity of the cartilage disease, disorder, or injury being treated. In embodiments, the compositions are administered as soon as possible after onset of symptoms, diagnosis or injury. In embodiments, compositions are administered more than one time following onset of symptoms, diagnosis or injury. In embodiments, where surgery is required, the cell-based compositions are administered at surgery. In embodiments, the compositions are administered at as well as after surgery. Such post-surgical administration may take the form of a single administration or multiple administrations.

In any method described herein, the cells or protein can be administered to the subject in combination with implantation of acellular scaffolds.

In embodiments, at least one additional agent may be combined with the cells or proteins described herein for administration to an individual according to any of the methods disclosed herein. Such agents may act synergistically with the cells of the invention to enhance the therapeutic effect. Such agents include, but are not limited to, growth factors, cytokines, chemokines, antibodies, inhibitors, antibiotics, immunosuppressive agents, steroids, anti-fungal agents, anti-viral agents or other cell types (i.e. stem cells or stem-like cells, for example AMP cells), extracellular matrix components such as aggrecan, versican hyaluronic acid and other glycosaminoglycans, collagens, etc. Inactive agents include carriers, diluents, stabilizers, gelling agents, delivery vehicles, ECMs (natural and synthetic), scaffolds, and the like. When the cells described herein are administered conjointly with other pharmaceutically active agents, even less of the cells may be needed to be therapeutically effective.

In embodiments, no additional active agent is combined with the cells or proteins described herein for administration to an individual.

### EMBODIMENTS

Embodiments contemplated herein include embodiments P1 to P14 following.

### Embodiment P1

An in vitro mesenchymal stem cell culture comprising a plurality of mesenchymal stem cells and an amount of a protein selected from chordin-like protein 1 (CHRDL1) and collagen VI effective to increase in vitro growth, expansion or survival of said plurality of mesenchymal stem cells relative to the absence of said protein.

### Embodiment P2

The mesenchymal stem cell culture of embodiment P1 comprising a liquid culture media.

### Embodiment P3

The mesenchymal stem cell culture of embodiment P2 wherein said protein is soluble in said liquid culture media.

### Embodiment P4

A method of increasing growth, expansion or survival of a plurality of mesenchymal stem cells, the method comprising contacting said plurality of mesenchymal stem cells with an effective amount of a protein selected from chordin-like protein 1 (CHRDL1) and collagen VI thereby increasing growth, expansion or survival of a plurality of mesenchymal stem cells.

### Embodiment P5

A method of administering a plurality of mesenchymal stem cells to a subject in need thereof, the method comprising: a. Contacting in vitro said plurality of mesenchymal stem cells with an effective amount of a protein selected from chordin-like protein 1 (CHRDL1) and collagen VI thereby increasing growth, expansion or survival of said plurality of mesenchymal stem cells; and b. administering said plurality of mesenchymal stem cells to said subject.

### Embodiment P6

The method of embodiment P5, wherein said subject is a in need of a transplantation procedure.

### Embodiment P7

The method of embodiment P6 further comprising transplanting additional biological tissue to said subject.

### Embodiment P8

The method of embodiment P5 wherein said subject is in need of cartilage growth such as cartilage repair.

### Embodiment P9

The method of embodiment P8 wherein said isolated mesenchymal stem cells differentiate to cartilage cells after said administering.

### Embodiment P10

The method of any one of embodiments P5-P9 further comprising administering a protein selected from chordin-like protein 1 (CHRDL1) and collagen VI.

### Embodiment P11

A method of increasing cartilage growth in a subject in need thereof, the method comprising administering an effective amount of a protein selected from chordin-like protein 1 (CHRDL1) and collagen VI.

### Embodiment P12

The method of embodiment P11 further comprising administering a plurality of mesenchymal stem cells.

### Embodiment P13

The method of any one of embodiments P11 or P12 wherein said administering is through injection.

### Embodiment P14

The method of embodiment P13 wherein said injection is provided at or near a cartilage-deficient site.

### EXAMPLES

### Example 1—Identification of Human Juvenile Chondrocyte-Specific Factors for Engineering Cartilage

Abstract.

Although regeneration of human cartilage is inherently inefficient, age is an important risk factor for Osteoarthritis (OA). Recent reports have provided compelling evidence that juvenile chondrocytes (from donors below 13 years of age) are more efficient at generating articular cartilage as compared to adult chondrocytes. However, the molecular basis for such a superior regenerative capability is not understood. In order to identify the cell-intrinsic differences between young and old cartilage, we have systematically profiled global gene expression changes between a small cohort of human neonatal/juvenile and adult chondrocytes. No such study is available for human chondrocytes although ‘young’ and ‘old’ bovine and equine cartilage have been recently profiled. Our studies have identified and validated new factors enriched in juvenile chondrocytes as compared to adult chondrocytes including secreted ECM factors Chordin-like 1 (CHRDL1) and Microfibrillar-associated protein 4 (MFAP4). Network analyses identified cartilage development pathways, epithelial to mesenchymal transition and innate immunity pathways to be overrepresented in juvenile-enriched genes. Finally, CHRDL1 was observed to aid the proliferation and survival of human bone-marrow derived mesenchymal stem cells (MSC) providing a mechanism for how young cartilage factors can potentially enhance stem cell function in cartilage repair.

Introduction.

Damage to articular cartilage is a common injury and can frequently lead to degenerative Osteoarthritis with aging. Due to its low cellularity and highly avascular nature, articular cartilage has a limited ability to heal and consequently cartilage injuries frequently require surgical intervention or lead to Osteoarthritis (OA). Currently employed regenerative treatments for cartilage defects involve microfracture of the subchondral bone to release bone marrow derived mesenchymal stem cells (MSCs) into the cartilage or cell-based techniques including autologous chondrocyte implantation (ACT) (reviewed in [1]). However, the repair tissue is often a biochemically and structurally inferior ‘fibrocartilage’ rather than articular-like cartilage[2]. The inability of mature cartilage to regenerate efficiently places a huge economic burden on the healthcare system. More than 10% of the US adult population was affected by OA in 2005 with hip and knee joint replacement surgery costing an estimated $42.3 billion in 2009 [3].

Clinically, it has been long known that cartilage repair can be effective in young patients while in adults, the tissue formed after injury or trauma is inferior to normal functioning cartilage. A concise study in rabbits clearly showed superior repair in young rabbits as compared to adult [4]. Similarly, an in utero cartilage defect study in lamb showed efficient repair [5]. More recent reports have demonstrated that human juvenile cartilage cells (from donors below 13 years of age) are more efficient at generating functional cartilage as compared to the adult cells [6,7]. Juvenile chondrocytes have also been demonstrated to modify adult chondrocytes in co-culture experiments [8,9] suggesting that paracrine factors secreted from the young chondrocytes may have a therapeutic benefit. Upon injection into the intervertebral disc of rats, juvenile chondrocytes have been seen to exhibit a more active remodeling process than controls [10], however, the use of human juvenile chondrocytes for regenerative purposes is likely to be limited by donor supply. Co-culture studies using a combination of juvenile chondrocytes with adipose-derived stem cells (ADSCs) to overcome this hurdle have yielded some promising results, suggesting that small numbers of juvenile chondrocytes in combination with ADSCs can be used to induce robust cartilage formation [11].

Although multiple studies have documented the superior potential of juvenile chondrocytes, the molecular basis of such superior regeneration has not been explored. Recent studies in bovine and equine young and adult cartilage have begun to address this question, however a precise study of the molecular factors and biological pathways that underpin these differences in humans has not yet been documented. Part of the challenge for the lack of such studies is the difficulty in obtaining healthy human cartilage especially from young donors. To address this, we have examined the gene expression profiles of a small cohort of human juvenile and adult chondrocytes, and of human mesenchymal stem cells (hMSCs) utilizing exon microarrays. Analyses of the global gene expression differences has allowed us to identify factors that distinguish juvenile chondrocytes from adult chondrocytes including paracrine factors secreted exclusively by the juvenile chondrocytes as compared to the adult chondrocytes. In addition, we explore the potential roles that the identified factors may play in the enhanced ability of juvenile chondrocytes for cartilage repair and regeneration.

Methods.

Chondrocyte isolation and culture. Adult (34 years) and juvenile articular chondrocytes (24 weeks fetus, 6 months, 18 months) were purchased from Lonza (CLONETICS™, Lonza Walkersville, Inc.) and cultured in Chondrocyte Growth Medium (CLONETICS CGM™, Lonza Walkersville, Inc.). Articular cartilage was harvested from grossly normal cartilage pieces discarded during notchplasty or debridement from patients undergoing anterior cruciate ligament (ACL) reconstruction with no history of OA symptoms (aged 18, 27 and 39 years old) and from OA patients undergoing total joint replacement (aged 64 and 79 years old) under approved Human Subjects Institutional Review Board protocols. Cartilage was dissected and the chondrocytes dissociated and cultured in high-density monolayers for limited passages, as described previously [12].

Human mesenchymal stem cells were purchased from Lonza (Lonza Walkersville, Inc.) (three samples from adult donors aged 19, 27 and 30 years old) and cultured in Mesenchymal Stem Cell Growth Medium (MSCGM™, Lonza Walkersville, Inc.).

Microarrays and Gene expression analyses. Total RNA was purified using the Qiagen RNeasy Plus Mini kit (Qiagen). For quantitative PCR, cDNA was synthesized using the High Capacity cDNA Reverse Transcription Kit (Applied Biosystems) and the USB Veriquest SYBR Green qPCR Master Mix (Affymetrix) was used with gene specific primers. All analysis was performed using the ddCT method and expression was normalized to GAPDH or 18S.

SYBR green primer sequences were as follows: Human GAPDH: F—TGTCCCCACTGCCAACGTGTC (SEQ ID NO:1), R AGCGTCAAAGGTGGAGGAGTGGGT (SEQ ID NO:2); CD24: F—AACTAATGCCACCACCAAGG (SEQ ID NO:3), R—AAGACGTTTCTTGGCCTGAG (SEQ ID NO:4); CHRDL1: F—TGCGAGTACAATGGGACAAC (SEQ ID NO:5), R—GTTGCCGATTCTGAAAGAGC (SEQ ID NO:6); :OMD F—GTGTCAGTGAATGCTTCTGTCC (SEQ ID NO:7), R—GAGTTGCTGAATGTGCATCG (SEQ ID NO:8); MFAP4: F—TGAGAACAACACGGCCTATG (SEQ ID NO:9), R—TCAAAGCCTGCCACAAAGAG (SEQ ID NO:10); :SHOX F—CAACGTGGAAAAGGCGTTAC (SEQ ID NO:11), R—TCCCCTTCAGAAACACAACC(SEQ ID NO:12).

For microarray expression analyses, purified RNA was run on a Human gene 1.0 ST array (Affymetrix). Two independent replicate samples for hMSCs and adult chondrocytes and three independent replicate samples for juvenile chondrocytes were run on Human Gene 1.0 ST Arrays (Affymetrix). Data analysis was performed using dChip [13] as described by the manual, and network analysis of differentially expressed genes was performed using MetaCore (Thomson Reuters).

Immunohistochemistry. Juvenile and adult chondrocytes were fixed in 4% paraformaldehyde (Sigma) then permeabilized in cold methanol (Sigma), before blocking in PBS containing 1% BSA, 10% FBS and 0.4% Triton X-100. Cells were incubated with primary antibody overnight (anti-CHRDL1 1:100 (Bioss USA) and anti-MFAP4 1:200 (Santa Cruz)). The following day cells were washed in PBS and incubated for 1 hour in secondary antibody (Alexa 594 goat anti-rabbit 1:250 (Invitrogen)) and cellular DNA was counterstained with DAPI (Life Technologies).

Treatment with Juvenile factors. Chondrocytes and hMSCs were plated at 1000 cells per well in duplicates in 48-well plates and cultured for 24 hours in complete medium. After 24 hours, cells were treated with control or medium containing 100 nM of recombinant human CHRDL1 (R&D Systems) or 25 nM of recombinant human MFAP4 (Abeam) or 100 nM of soluble Col VI (BD Biosciences), with media and recombinant protein replacement every day for 4 days.

Cell proliferation assay. Cell viability was assayed daily in the recombinant protein treatment assays with the PrestoBlue Cell Viability Reagent (Life Technologies) following the manufacturer's instructions. PrestoBlue reagent was added to the cell culture medium, and the cells were incubated at 37° C. for 30 min. Fluorescence intensity of the PrestoBlue was measured at 690 nm (650 nm excitation wavelength) with a microplate reader (Molecular Devices). Fluorescence level was expressed in Relative Fluorescence Units (RFU).

Results.

Juvenile chondrocytes have gene expression patterns distinct from both adult chondrocytes and MSC. Striking phenotypic and functional differences have been previously reported between juvenile and adult human chondrocytes [6,14], however to date no differential molecular regulators or pathways have been identified for the young and adult chondrocytes. Without wishing to be bound by any theory, it is believe that a major difficulty for such analyses is the paucity of normal human cartilage samples. As a first attempt to identify juvenile chondrocyte-specific factors that provide them with the characteristics different from adult chondrocytes, we have utilized a small cohort of juvenile and adult chondrocytes consisting of juvenile chondrocytes isolated from three different donors and adult chondrocytes isolated from two different donors. In addition, these juvenile and adult chondrocyte samples were previously characterized by us [14] and demonstrated the characteristic functional differences such as increased ECM production by the juvenile chondrocytes compared to adult chondrocytes. To investigate the molecular differences between the juvenile and adult chondrocytes, we utilized exon microarrays to determine their global gene expression profiles. For the microarray analysis, total RNA was extracted from chondrocytes after limited cell culture and expansion (1-2 passages) in high-density monolayers to prevent dedifferentiation, as described previously [15]. Human Gene 1.0 ST Arrays (Affymetrix) were utilized and data analysis was performed using dChip [13].

To identify the differentially expressed genes in the juvenile chondrocytes, we first compared them to the adult chondrocytes (schematic FIG. 1A). Genes were ranked according to the average fold change in transcript expression in all the juvenile chondrocyte samples averaged together compared to the transcript expression in the averaged adult chondrocyte samples. Analysis of genes with a 1.5-fold or greater increase in expression identified 597 number of genes that were upregulated in the juvenile chondrocytes compared to the adult chondrocytes. The top 20 genes are tabulated in FIG. 1B. Network analyses of all the genes with a 1.5-fold or greater increase in expression in juvenile versus adult chondrocytes interestingly identified pathways already implicated in early chondrocyte development including epithelial to mesenchymal transition and cartilage development pathways (FIG. 1C). We also performed network analysis on the genes with a 1.5-fold or greater decrease in the juvenile chondrocytes when compared to the adult chondrocytes which, as expected, revealed pathways known to be upregulated in mature chondrocytes.

Of the genes identified as having a 1.5-fold or greater increase in expression in the juvenile chondrocytes when compared to the adult, we focused on the genes with the most differential expression (the top 20 genes, ranked by fold change (FIG. 1B)). This list of unique juvenile chondrocyte genes included several genes that had previously been implicated in chondrocyte or extracellular matrix function. Initially, we chose to further investigate paracrine factors that were differentially secreted by the juvenile chondrocytes. These factors included chordin-like 1 (CHRDL1) a known BMP-antagonist [16], osteomodulin (OMD) a proteoglycan that is highly expressed in bone and cartilage progenitor cells [17], and microfibrillar associated protein 4 (MFAP4) [18] an extracellular matrix protein known to bind collagen.

Next, we utilized human bone-marrow derived mesenchymal stem cells (hMSC) from two different donors, to investigate whether the juvenile chondrocytes share any stem cell-like factors with the hMSC. We analyzed genes that had a 1.5-fold or greater increase in expression in the juvenile chondrocytes when compared to the hMSCs. Network analysis of the genes with a 1.5-fold upregulation in the juvenile chondrocytes revealed several pathways involved in cell cycling and repair of DNA damage. Network analysis of the genes with a 1.5-fold or greater decrease in the juvenile chondrocytes when compared to the hMSCs identified pathways related to other lineages, indicating the commitment of the juvenile chondrocytes to the chondrogenic lineage as opposed to the multipotent hMSCs.

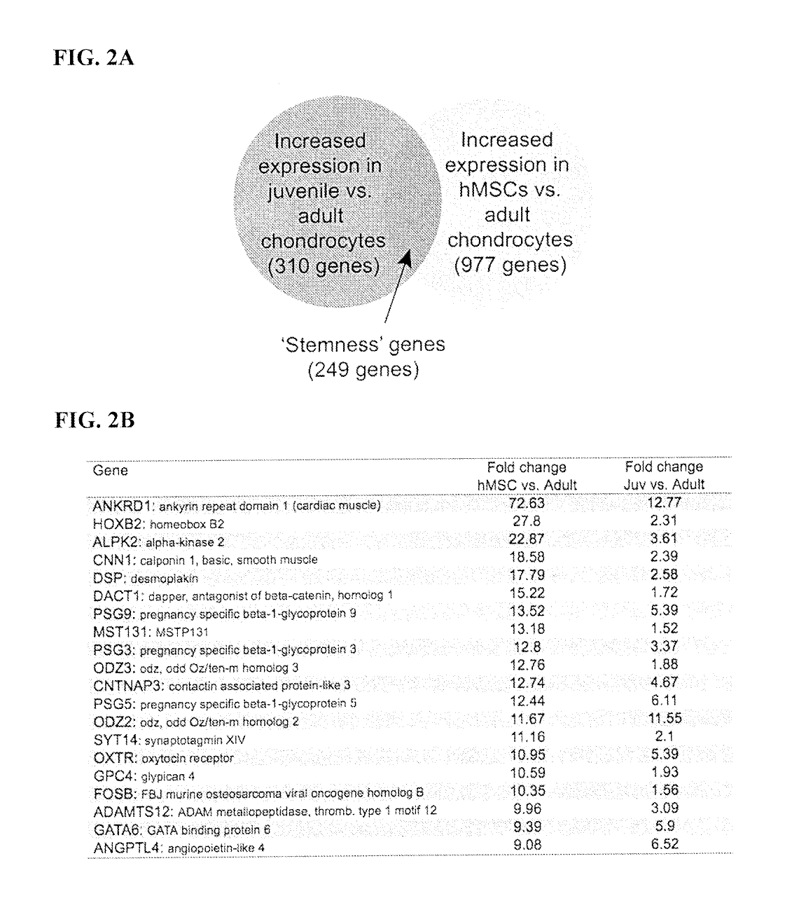

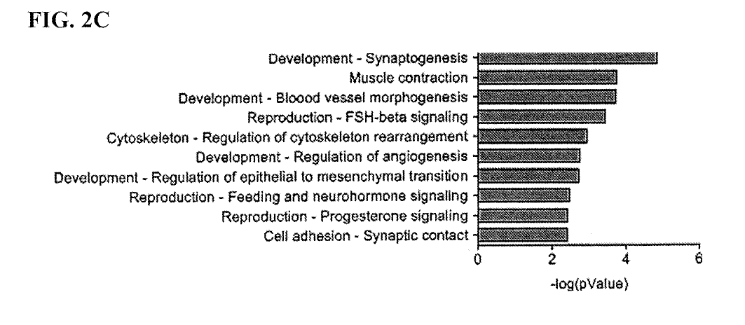

Next, we analyzed the subset of genes that are upregulated in both juvenile chondrocytes and hMSCs compared to adult chondrocytes and thus may represent genes indicative of stem-cell-like qualities or ‘stemness’, in juvenile chondrocytes (FIG. 2A). The term “stemness” refers to qualities of a cell (e.g., morphology, expressed proteins, capacity for differentiation, and the like) which are indicative, at least in part, of stem cells. We identified 249 genes that are upregulated by 1.5-fold or greater in both juvenile chondrocytes and hMSCs when compared to adult chondrocytes (shown in schematic in FIG. 2A and the top 20 are listed in FIG. 2B). To identify the pathways these genes represented, we performed network analyses that identified multiple developmental and reproductive pathways (FIG. 2C), suggesting that there is possibly a stem cell-like population or characteristics present in juvenile chondrocytes that may be responsible for their high regeneration potential. Common genes so identified included HoxB2, a family member of homeo-box genes known to play regulatory roles in vertebrate patterning [19] and ADAMTS12, a metalloproteinase implicated in growth plate development as well as cartilage degeneration in arthritis [20].

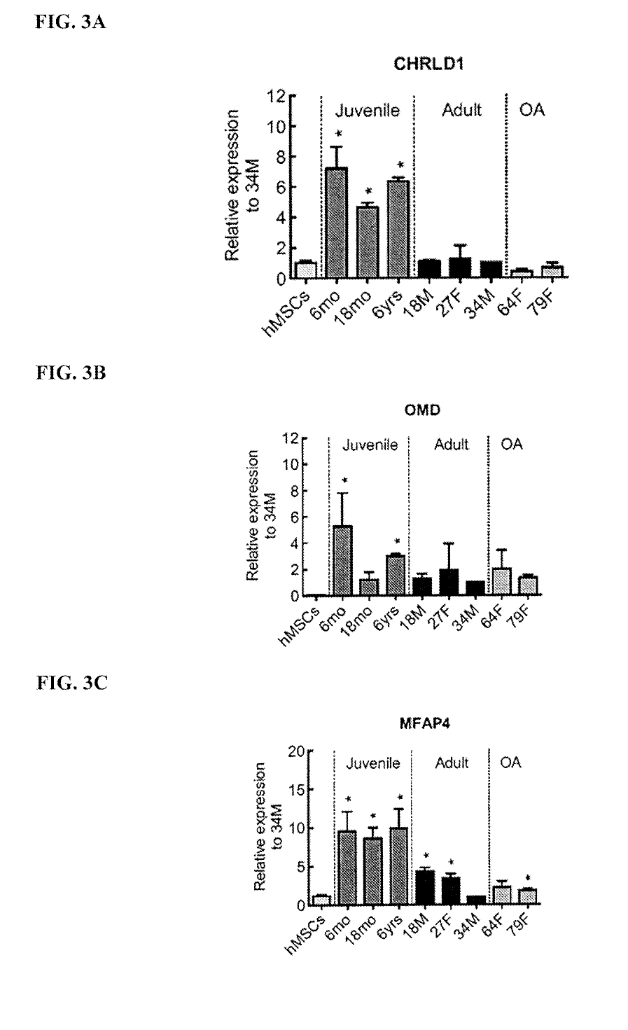

Validation of Juvenile chondrocyte-specific factors. To validate the findings from the microarray study and to explore the factors of interest further, we examined the mRNA levels of the putative juvenile factors by quantitative PCR (qPCR). We used a range of samples including additional juvenile and adult chondrocyte besides the ones used for the initial microarray experiments for these validation studies. We also included samples from osteoarthritic (OA) patients to test whether the identified juvenile factors are altered in the diseased state since some embryonic gene transcripts (Col2a1, for example) are known to get upregulated in OA chondrocytes. The quantitative PCR data was in good agreement with the microarray data and identified trends in the expression patterns of these factors (FIGS. 3A-3C). Compared to the expression in hMSCs, CHRDL1 was upregulated 5-fold or more in all juvenile samples examined, whereas the adult and OA samples showed no change in expression (FIG. 3A). OMD gene expression was somewhat more variable; elevated levels were observed in the juvenile chondrocytes compared to hMSC but some adult and OA samples also showed moderate upregulation (FIG. 3B). MFAP4 was upregulated approximately 10-fold in all the juvenile chondrocytes, and showed progressive decreases in expression levels with age in adult chondrocytes (FIG. 3C).

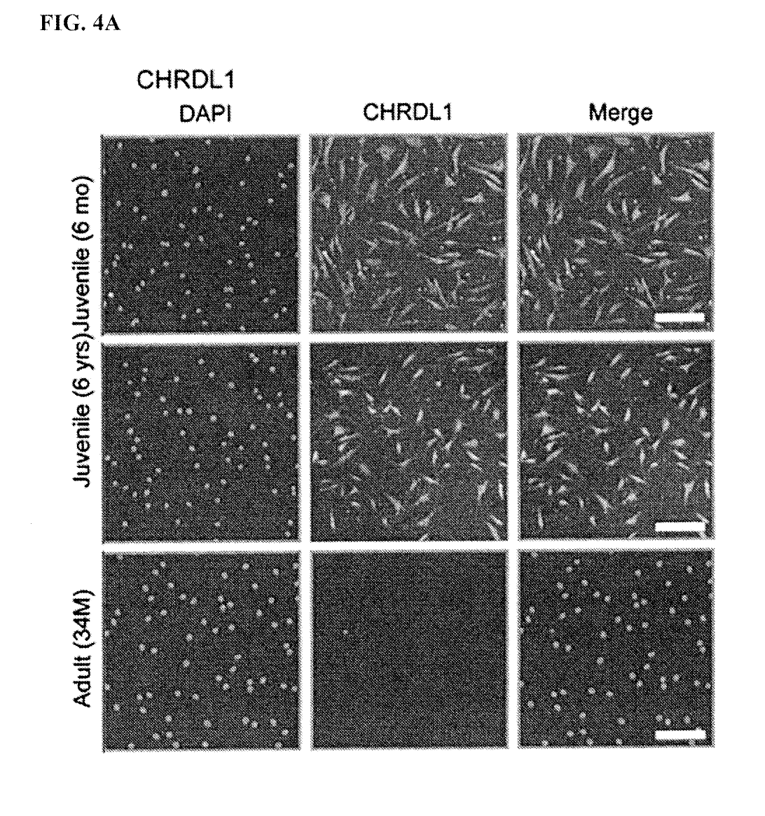

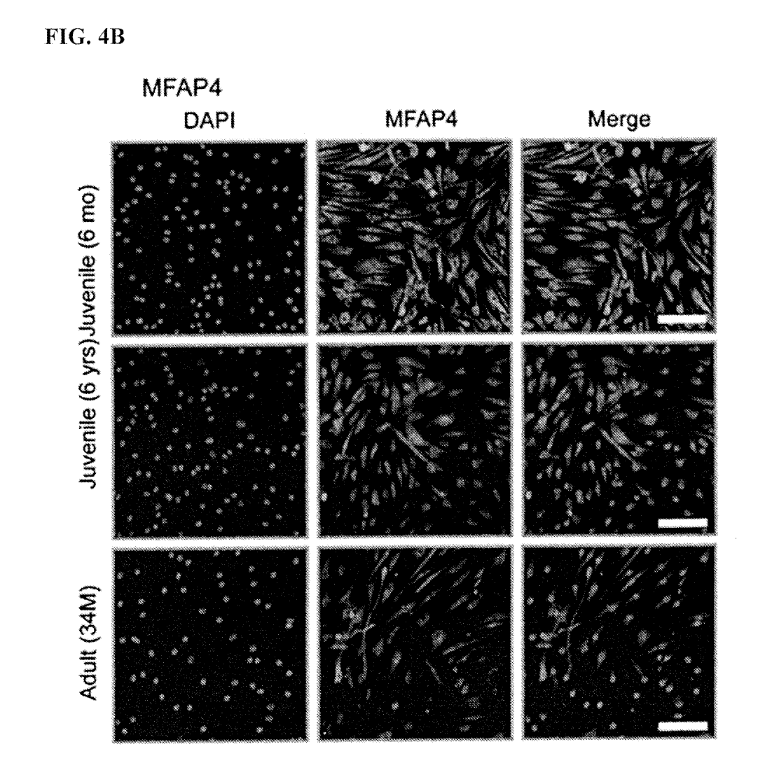

MFAP4 shows an age-dependent decrease while CHRDL1 is absent in adult chondrocytes. To assess whether the juvenile chondrocyte specific factors we identified through our mRNA studies were upregulated at the protein level, we performed immunostaining on juvenile and adult chondrocytes. Since the expression levels of OMD were somewhat variable, we omitted this factor from further investigations. We observed that CHRDL1 was clearly expressed at the protein level in juvenile chondrocytes whereas expression was completely absent in the adult chondrocytes (FIG. 4A). On the other hand, MFAP4 displayed more intense staining in the juvenile chondrocytes compared to the adult, but a decline in expression was observed in the adult samples (FIG. 4B), in good correlation with the mRNA expression data (FIGS. 3A-3C).

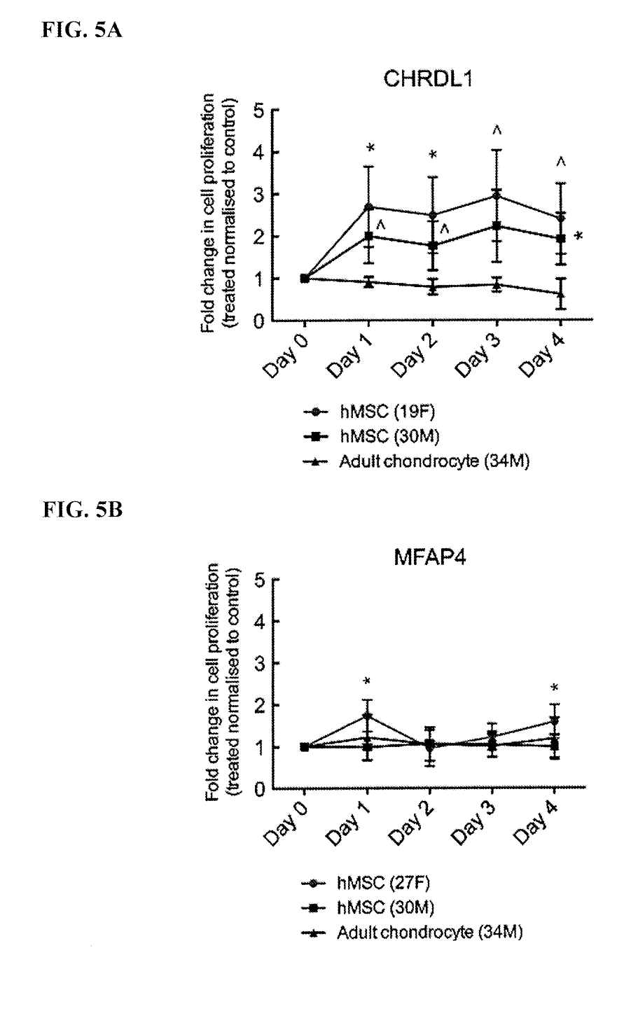

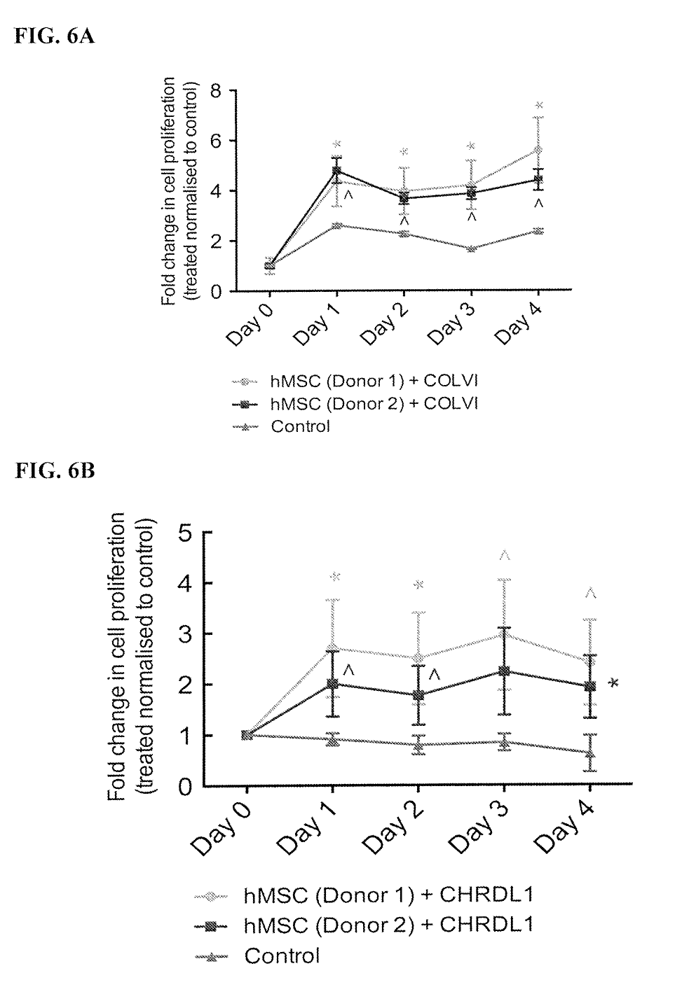

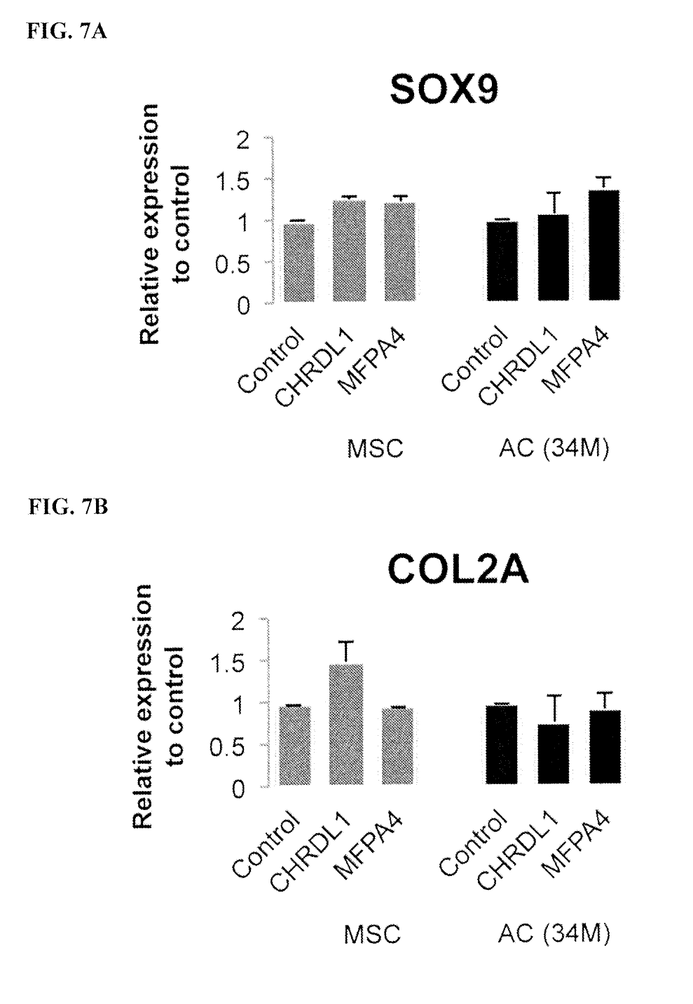

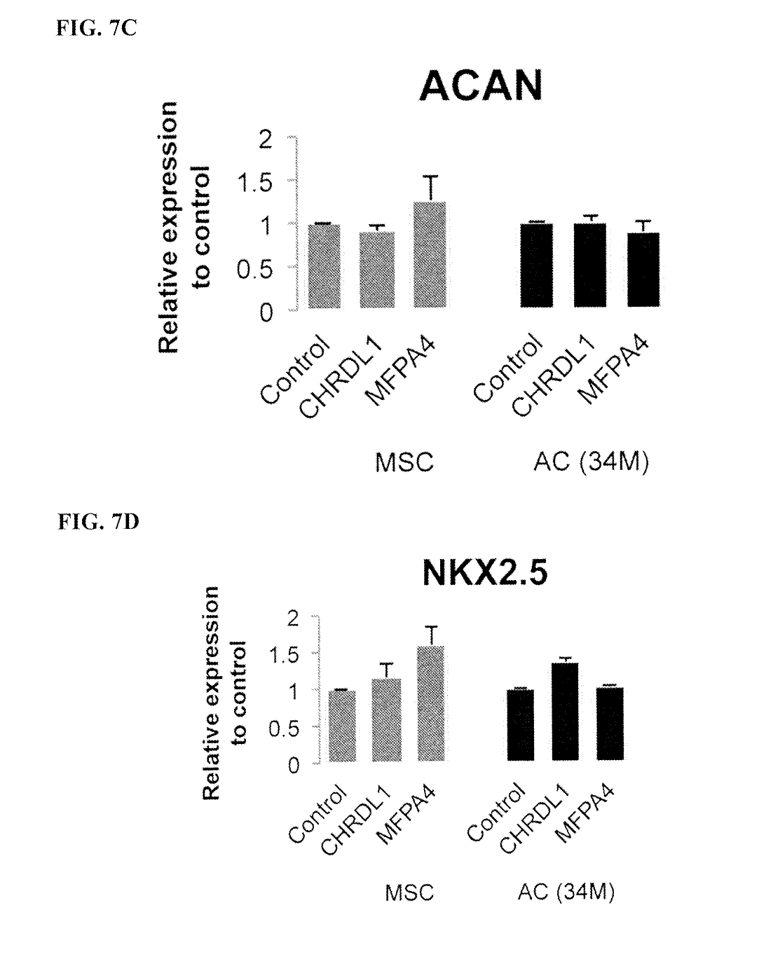

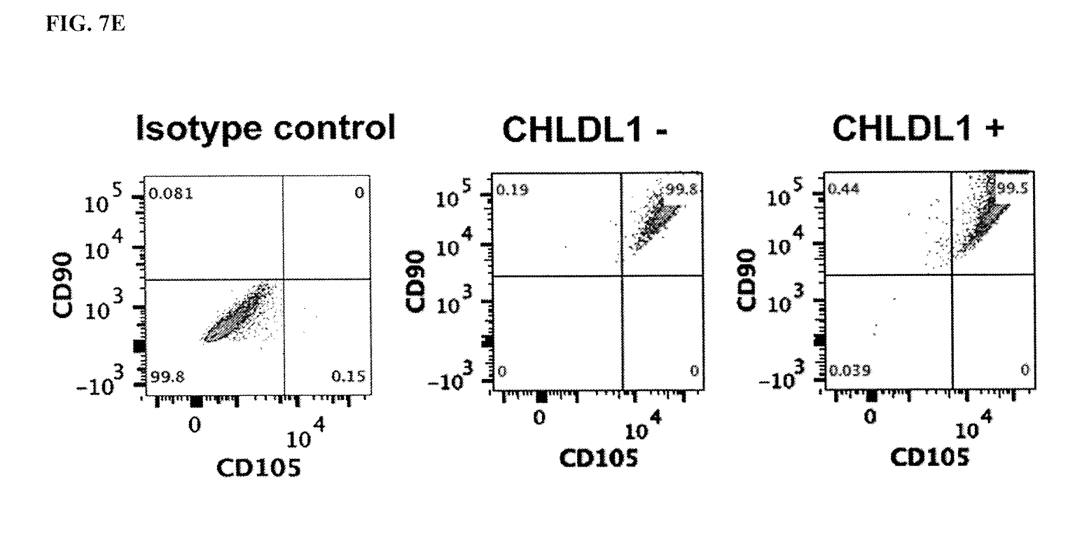

CHRDL1 stimulates an increase in hMSC proliferation but does not affect proliferation of adult chondrocytes. Next, we explored a possible mode of action of CHRDL1 and MFAP4 in young cartilage, specifically testing if either of these factors promoted stem cell survival or proliferation. We exposed hMSCs and adult chondrocytes to either soluble recombinant human CHRDL1 or MFAP4 and observed the effect on the proliferation of hMSCs and adult chondrocytes. When cultured for 4 days in the presence of CHRDL1, hMSCs showed a 3-4-fold increase in cell proliferation, whereas no effect was seen on adult chondrocytes (FIG. 5A). Neither the hMSCs nor the adult chondrocytes showed an increase in proliferation in response to MFAP4 treatment (FIG. 5B). As disclosed in FIGS. 6A-6B, contacting hMSC with Collagen VI (Col VI) or CHRDL1 stimulate proliferation of hMSC.

Discussion.

Although the higher repair potential of juvenile chondrocytes has been reported in the last few years [6,7] the molecular regulators responsible are unknown. Detailed characterization of juvenile and adult chondrocytes is required to dissect the underlying biological differences that lead to the observed increased regenerative capability of juvenile chondrocytes. Identification of the human juvenile chondrocyte-specific factors will not only advance the fundamental understanding of the age-associated loss of regeneration potential but also provide new strategies for enhancing cartilage regeneration and for engineering superior cartilage tissue. Recently, two studies have described global gene expression profiling of juvenile and adult cartilage in bovine and equine tissues respectively [21,22]. The study in bovine articular cartilage identified genes related to cartilage growth and expansion (COL2A1, COL9A1, MMP2, MMP14 and Tom to be upregulated in bovine juvenile chondrocytes, whereas structural genes (COMP, FN1, TIMP1, TIMP3 and BMP2) were upregulated in bovine adult cartilage [22]. The equine study utilized genome-wise RNA sequencing (RNA-seq) and identified expression of multiple ECM genes including collagen types, biglycan, asporin and various matrilins to be decreased in old cartilage while the expression of Col10a1 and col25a1 was increased [21]. Another interesting difference was a reduction in multiple Wnt pathway factors with an increase in the Wnt antagonist DKK1 in the older cartilage [21].

Limitation of obtaining normal cartilage samples has generally precluded such studies in human tissues. We had previously established quantitative functional methods to distinguish juvenile and adult chondrocytes that were consistent with previously reported characteristics such as increased ECM production by the juvenile chondrocytes compared to adult chondrocytes. The small cohort of juvenile and adult chondrocyte samples utilized retained the expected functional differences. The average profile of the juvenile and adult chondrocytes clearly showed distinct gene expression patterns although the general chondrogenic markers (for example Sox9, Col2a1, Agan) were expressed in both populations as described earlier [14].

Many of the genes that were highly expressed in juvenile chondrocytes as compared to the adult chondrocytes were secreted extracellular matrix (ECM) factors. Besides the known ECM factors like col2a1 and col6 that are known to be enriched in juvenile cartilage as compared to adult cartilage, we identified new factors like Chordin-like 1 (CHRDL1) and MFAP4 that were preferentially secreted by juvenile chondrocytes. CHRDL1 is interesting since chordin is a known BMP antagonist [23]. The family member Chordin-like2 (CHRDL2) has been identified as being expressed exclusively in the cartilage of the developing joint and connective tissue in reproductive organs [16]. The extracellular protein MFAP4 was upregulated in juvenile cartilage at the protein level and there was a notable decrease with aging, unlike the protein expression of CHRDL1 that was expressed only in the juvenile chondrocytes with little or no expression observed in the adult chondrocytes. Our studies have therefore identified new molecular factors that are enriched in juvenile human chondrocytes and undergo an age-dependent decline.

Next, we addressed a molecular basis for the enhanced regenerative potential of juvenile human chondrocytes. Without wishing to be bound by any theory, it is believe that one possible reason can be an enrichment of stem cell-like factors in juvenile chondrocytes that allows increased proliferation and hence more ECM production than the adult chondrocytes. Another possible explanation for a higher regenerative potential for juvenile cartilage could be the secreted paracrine factors that can support stem cell function, survival or differentiation better than the adult cartilage. To test the first possibility, we tested whether some of the mesenchymal stein cell factors are enriched in the juvenile chondrocytes. We identified 249 genes that are commonly upregulated in juvenile chondrocytes and hMSCs in comparison to adult chondrocytes. Network analyses indicated that these genes were involved in reproductive and developmental pathways suggesting that juvenile chondrocytes potentially retain some mesenchymal stem/progenitor factors that contribute to their regenerative potential. These common genes included HoxB2, a family member of homeo-box genes known to play regulatory roles in vertebrate patterning¹⁹ and ADAMTS12, a metalloproteinase implicated in growth plate development as well as cartilage degeneration in arthritis [20].

To test the ability of the newly identified juvenile factors to support stem cell function, we tested their effect on the proliferation of hMSCs. While MFAP4 did not show any significant effect on the proliferation of hMSCs, CHRDL1 showed a remarkable stimulation of hMSC proliferation. These observations were confirmed in hMSC derived from two independent donors. Hence, it remains possible that the juvenile cartilage has a dual advantage towards cartilage regeneration—the intrinsic increase in the growth and ECM generation capability of the juvenile chondrocytes as well as secretion of paracrine factors by juvenile chondrocytes that stimulate stem cell function.

In summary, these studies provide a first global gene expression snapshot of juvenile chondrocytes in comparison to adult chondrocytes and identify new juvenile chondrocyte factors and their potential stimulatory effects on stem cell function. It is believed that the identified factors can be useful for the stem cell based tissue engineering strategies for cartilage regeneration.

### REFERENCES (EXAMPLE 1)

[1] Huey, D. J., Hu, J. C. & Athanasiou, K. A. Unlike bone, cartilage regeneration remains elusive. Science 338, 917-921, doi:10.1126/science.1222454 (2012); [2] Falah, M., Nierenberg, G., Soudry, M., Hayden, M. & Volpin, G. Treatment of articular cartilage lesions of the knee. International Orthopaedics 34, 621-630, doi:10.1007/s00264-010-0959-y (2010); [3] Murphy, L. & Helmick, C. G. The impact of osteoarthritis in the United States: a population-health perspective. AJN The American Journal of Nursing 112, S13-S19 (2012); [4] Wei, X. & Messner, K. Maturation-dependent durability of spontaneous cartilage repair in rabbit knee joint. J Biomed Mater Res 46, 539-548 (1999); [5] Namba, R. S., Meuli, M., Sullivan, K. M., Le, A. X. & Adzick, N. S. Spontaneous repair of superficial defects in articular cartilage in a fetal lamb model. J Bone Joint Surg Am 80, 4-10 (1998); [6] Adkisson, H. D., Gillis, M. P., Davis, E. C., Maloney, W. & Hruska, K. A. In vitro generation of scaffold independent neocartilage. Clin Orthop Relat Res, S280-294 (2001); [7] Adkisson, H. D. t. et al. The potential of human allogeneic juvenile chondrocytes for restoration of articular cartilage. Am J Sports Med 38, 1324-1333, doi:10.1177/0363546510361950 (2010); [8] Bonasia, D. E. et al. Cocultures of adult and juvenile chondrocytes compared with adult and juvenile chondral fragments: in vitro matrix production. Am J Sports Med 39, 2355-2361, doi:10.1177/0363546511417172 (2011); [9] Acosta, F. L., Jr. et al. Porcine intervertebral disc repair using allogeneic juvenile articular chondrocytes or mesenchymal stem cells. Tissue Eng Part A 17, 3045-3055, doi:10.1089/ten.tea.2011.0229 (2011); [10] Kim, A. J. et al. Juvenile chondrocytes may facilitate disc repair. Open Tissue Eng Regen Med J 3 (2010); [11] Lai, J. H., Kajiyama, G., Smith, R. L., Maloney, W. & Yang, F. Stem cells catalyze cartilage formation by neonatal articular chondrocytes in 3D biomimetic hydrogels. Scientific Reports 3, 1-9, doi:10.1038/srep03553 (2013); [12] Snickers, Y. H., Weinans, H., Bierma-Zeinstra, S. M., van Leeuwen, J. P. & van Osch, G. J. Animal models for osteoarthritis: the effect of ovariectomy and estrogen treatment—a systematic approach. Osteoarthritis Cartilage 16, 533-541, doi:10.1016/j.joca.2008.01.002 (2008); [13] Li, C. & Wong, W. H. Model-based analysis of oligonucleotide arrays: expression index computation and outlier detection. Proceedings of the National Academy of Sciences 98, 31-36 (2001); [14] Smeriglio, P. et al. Comparative Potential of Juvenile and Adult Human Articular Chondrocytes for Cartilage Tissue Formation in Three-Dimensional Biomimetic Hydrogels. Tissue Eng Part A, doi:10.1089/ten.TEA.2014.0070 (2014); [15] Taylor, S. E., Smeriglio, P., Dhulipala, L., Rath, M. & Bhutani, N. A global increase in 5-hydroxymethylcytosine levels marks osteoarthritic chondrocytes. Arthritis Rheumatol 66, 90-100, doi:10.1002/art.38200 (2014); [16] Nakayama, N. et al. A novel chordin-like BMP inhibitor, CHL2, expressed preferentially in chondrocytes of developing cartilage and osteoarthritic joint cartilage. Development 131, 229-240, doi:10.1242/dev.00901 (2004); [17] Liu, T. M. et al. Identification of Common Pathways Mediating Differentiation of Bone Marrow- and Adipose Tissue-Derived Human Mesenchymal Stem Cells into Three Mesenchymal Lineages. STEM CELLS 25, 750-760, doi:10.1634/stemcells.2006-0394 (2006); [18] Zhao, Z. et al. The gene for a human microfibril-associated glycoprotein is commonly deleted in Smith-Magenis syndrome patients. Hum Mol Genet 4, 589-597 (1995); [19] Gouveia, A., Marcelino, H. M., Goncalves, L., Palmeirim, I. & Andrade, R. P. Patterning in time and space: HoxB cluster gene expression in the developing chick embryo. Cell Cycle 14, 135-145, doi:10.4161/15384101.2014.972868 (2015); [20] Nah, S. S. et al. Association of ADAMTS12 polymorphisms with rheumatoid arthritis. Mol Med Rep 6, 227-231, doi:10.3892/mmr.2012.867 (2012); [21] Paters, M., Liu, X. & Clegg, P. Transcriptomic signatures in cartilage ageing. Arthritis Res Ther 15, R98, doi:10.1186/ar4278 (2013); [22] Liu, H. et al. Enhanced tissue regeneration potential of juvenile articular cartilage. Am J Sports Med 41, 2658-2667, doi:10.1177/0363546513502945 (2013); [23] Zhang, D. et al. A role for the BMP antagonist chordin in endochondral ossification. J Bone Miner Res 17, 293-300, doi:10.1359/jbmr.2002.17.2.293 (2002);

### Example 2. Soluble Collagen VI Treatment Enhances Mesenchymal Stem Cell Expansion for Engineering Cartilage

Abstract. Bone Marrow-derived Mesenchymal Stem Cells (BM-MSC) are an attractive source for cell-based therapies in cartilage injury owing to their efficient differentiation into chondrocytes. Additionally, MSC have been shown to be immune-suppressive and are being tested as inflammatory modulators in Osteoarthritis (OA) (Glenn, 2014); however, their clinical use is hampered by a scarcity of cells leading to compromised efficacy. While expansion of MSC ex-vivo can potentially overcome the scarcity of cells, current methods lead to a rapid loss of the stem cell properties. Identification and development of biologics to enhance MSC expansion for cartilage repair, while maintaining their stem and functional properties, is a critical unmet need in Orthopedics. In this study, we report soluble collagen VI as a potential biologic that can expand the MSC population while maintaining their cartilage differentiation potential.

Introduction.

Osteoarthritis (OA) is a multifactorial disease that affects the articular cartilage causing deterioration of the joint function. Cartilage injuries and trauma are difficult to repair even in young adults due to the poor regenerative potential of the cartilage tissue, and greatly accelerates OA development³⁻⁶. Current treatments of OA are mainly palliative and aim at pain and symptoms management rather than disease modification. Lack of any disease modifying OA drugs (DMOADs) calls for new and effective therapies to repair and regenerate damaged articular cartilage, and to restore joint homeostasis to delay OA progression [8].

Current cell based clinical therapies include the use of autologous chondrocytes or autologous cells from mesenchymal tissues. Stimulation of the endogenous stem cell populations to repair cartilage injuries through microfracture leads to inefficient regeneration and formation of fibrocartilage tissue rather than hyaline cartilage. Autologous articular chondrocytes implantation (ACI) has been clinically approved but the applications have been marred by a paucity of cells and production of functionally inferior fibrocartilage. Mesenchymal stem cells (MSCs) and adipose-derived stem cells (ADSCs) have been extensively investigated to engineer and repair cartilage in basic and early translational studies. Besides their ability to differentiate towards multiple cell types including chondrocytes, bone and adipocytes, the beneficial effects of MSC in secretion of anti-inflammatory factors that mitigate inflammation has also been implicated for treatment of multiple diseases. The role of inflammation in the development of OA is being increasingly recognized [52]. As such, the beneficial anti-inflammatory effects of MSC in prevention or delay of the onset of OA is of substantial interest [53]. Currently, MSC are being explored as a treatment modality for multiple disease states in hundreds of Phase 1 and 2 clinical trials [9]. A major barrier to clinical translation has been a paucity of these adult stem cells. While expansion of MSCs ex-vivo can solve the scarcity of the cell source, current methods lead to a rapid loss of the stem cell properties and potency. Therefore, identification and development of biologics to enhance MSC expansion for cartilage repair, while maintaining their stemness and functional properties has a high clinical relevance.

It is widely accepted that both the extra-cellular matrix (ECM) and the peri-cellular matrix (PCM) play a critical role in cartilage function especially for maintaining its biochemical and biomechanical properties [5]. Our recent studies have demonstrated ECM proteins to be a major differential between juvenile and adult cartilage suggesting that the ECM interactions with the chondrocytes also regulate the regenerative capacity of cartilage (ref). Therefore, the emerging understanding of the crosstalk between ECM components and chondrocytes is required and should be taken in consideration for cartilage tissue engineering. Collagen VI (Col VI) is a major component of the chondrocyte PCM consisting of three major α-chains, α1, α2 and α3, along with alternate subunits α4, α5 or α6 that can substitute for α3 [7,8]. We have previously demonstrated that soluble Collagen VI (Col VI) treatment can increase the number of human chondrocytes after short-term treatment without adversely affecting their ability to generate cartilage. As an extension of that study, we wanted to interrogate the effects of Col VI on developmentally early mesenchymal populations.

Methods.

hMSCs culture. Human mesenchymal stem cells from two donors (19 years old, Male and 30 years old, Female) were purchased from Lonza (CLONETICS™, Lonza Walkersville Walkersville, USA) and cultured in monolayer using Mesenchymal Stem Cell Growth Medium (MSCGM™, Lonza) as per manufacturer instructions. Pellet formation was induced by gentle centrifugation of 1 million cells per pellet in a 15 ml conic tube. After 24 hours, chondrogenic differentiation of the pellets was induced in Chondrogenic media for 21 days.

Collagen VI treatment and cell growth. Collagen VI (BD 354261) (RD Biosciences) was dissolved in a 1.25 mM Tris solution. hMSCs were plated in monolayers at 1000 cells per well in duplicates in 96-well plates and cultured for 24 hours in complete medium. After 24 hours, cells were treated with control Tris or medium containing 2.5 μg/ml of recombinant human Collagen VI, with media and recombinant protein replacement every day for 4 days. Cell viability was assayed daily with the PrestoBlue Cell Viability Reagent kit (Life Technologies, Carlsbad, Calif.) as previously described (Smeriglio et al.). Fluorescence intensity of the PrestoBlue reagent reduced by living cells was measured at 690 nm (650 nm excitation wavelength) with a microplate reader (Molecular Devices).

Flow Cytometry. Cells were dissociated to a single-cell suspension using TrypLE solution, fixed in BD Cytofix buffer for 20 minutes at room temperature and permeabilized with BD Permeabilization/Wash buffer at 1×10⁷ cells per 1 mL for 10 minutes. Cells were stained with primary antibodies (mouse anti-human Sox9, anti-human CD44-PE/Cy7, anti-human CD90-Alexa647, BD bioscience and anti-human CD105) for 30 minutes at the concentration suggested by the manufacturer. Secondary antibodies (donkey anti-mouse IgG Alexa 488) were diluted by 1:250. Cells were scanned using a LSR II flow cytometer and analyzed with Flowjo software.

Gene expression analyses. For the monolayer cultures, total RNA was extracted using the RNeasy mini kit (Qiagen, Valencia, Calif.) and for pellet cultures, total RNA was obtained using TRIzol (Invitrogen, Carlsbad, Calif.). RNA from each sample was reversed transcribed using the High Capacity cDNA Reverse Transcription Kit (Applied Biosystems) and quantitative PCR was performed using TaqMan gene-specific expression arrays for Type II collagen—chain alpha 1 (Col2a1), Sox9, Aggrecan (Acan) and metalloproteinase 13 (MMP13) with a universal mastermix (Applied Biosystems). Gene expression levels were normalized internally to GAPDH. The relative expression levels were determined using the ΔCt method (CT gene of interest—CT internal control—GAPDH) and 2^{-ΔCt} equation is calculated as described by Schmittgen and Livak [20] and plotted.

Immunohistochemistry. hMSC pellets were fixed in 4% paraformaldehyde (Sigma) then permeabilized in cold methanol (Sigma), before blocking in PBS containing 1% BSA, 10% FBS and 0.4% Triton X-100. Cells were incubated with the following primary antibodies overnight: Col2a1 (Abeam; 1:100), Aggrecan (1:500), Col6a1 (Santa Cruz, 1:100) at the specified dilutions. The antibody anti-Aggrecan used for this study was a kind gift from. Prof. R. L. Smith [14]. The following day cells were washed in PBS and incubated for 1 hour in secondary antibody (Alexa 594 goat anti-rabbit 1:250 (Invitrogen)) and cellular DNA was counterstained with DAPI (Life Technologies).

Biochemical analyses. For the cell-hydrogel constructs (n=3) DNA and sulfated glycosaminoglycan (sGAG) production were quantified as follow. The hydrogels were lyophilized and digested in papainase solution (Worthington Biochemical, Lakewood, N.J.) at 60° C. for 16 hours [18,21]. DNA content was measured using the PicoGreen assay (Molecular Probes, Eugene, Oreg.) with Lambda phage DNA as standard. sGAG content was quantified using the 1,9-dimethylmethylene blue (DMMB) dye-binding assay with shark chondroitin sulfate (Sigma, St. Louis, Mo.) as standard [22]. GAG content of the acellular hydrogels was determined as a negative control, and subtracted from the amount of GAG released by the encapsulated cells during the 3 weeks of culture.

Results.

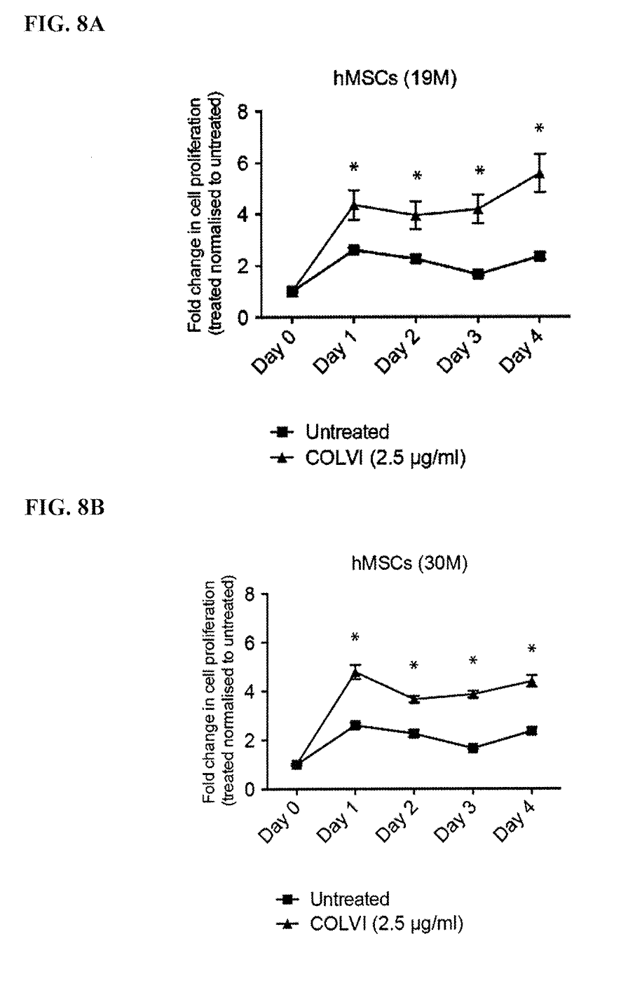

Collagen VI (Col VI) treatment enhances hMSC proliferation. In the present study, we aimed to study the direct effect of soluble Col VI on human bone-marrow derived mesenchymal stem cells (hMSC) fate and function. A quantitative fluorescence based assay reflecting the metabolic activity of live cells (Prestoblue, Life technologies) was used to measure cell growth, as described previously (ref). In this assay, the reagent resazurin is non-fluorescent but is converted to fluorescent resorufin in the reducing environment of living cells, allowing a quantitative measurement of relative cell numbers. The advantage of this reagent is both high sensitivity allowing detection of relatively low cell numbers reproducibly as well as minimal toxicity such that the reagent can be used at regular intervals during continuous cell culture. As shown in FIGS. 8A-8B, we observed a significant increase in cell proliferation in hMSC from two different healthy donors (19 and 30 years old, both males) upon treatment with recombinant Col VI (2.5 ug/ml) for 4 days compared to vehicle treated controls. The hMSC were plated at a cell number of 2000 and their cell growth was monitored every day in the absence or presence of Col VI with the fluoresence assay for 4 days. Soluble Col VI (2.5 μg/ml) was replenished daily. We observed a significant 2-3 fold increase in the number of both hMSC samples upon Col VI treatment.

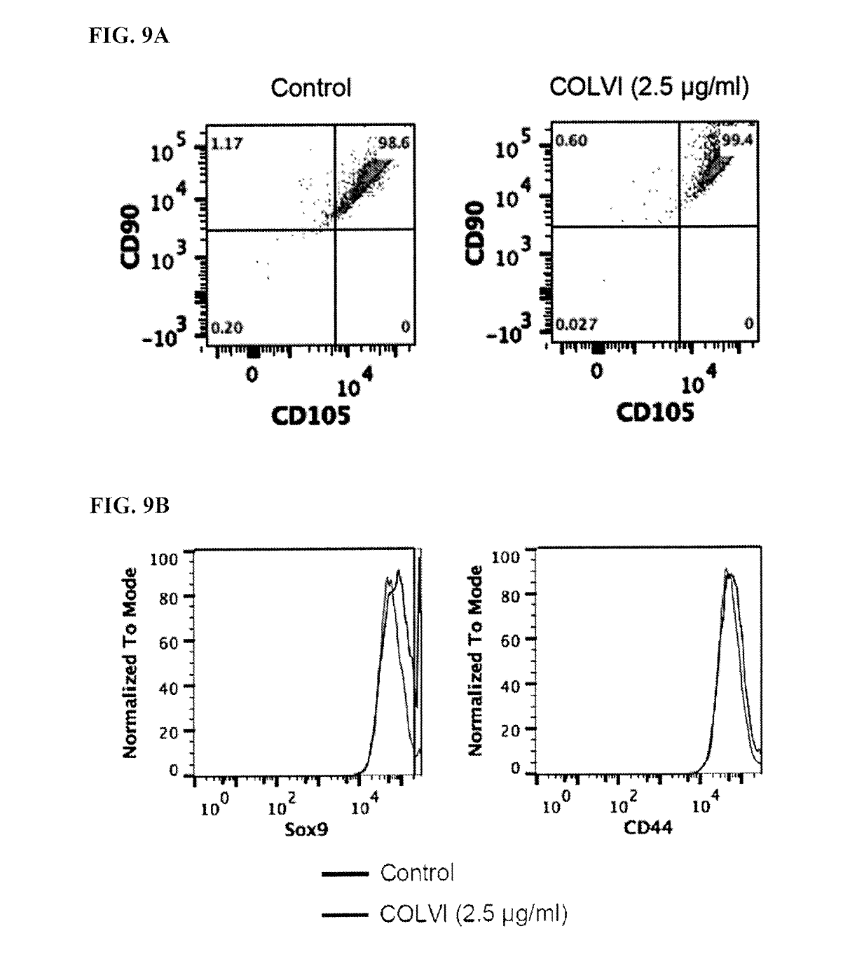

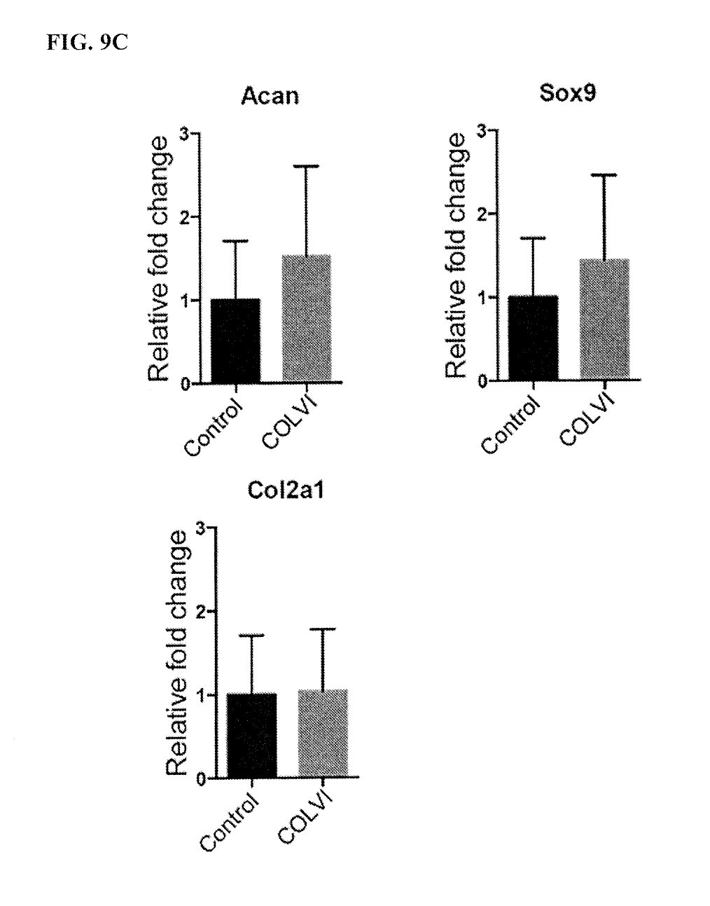

Col VI treatment maintains Mesenchymal Stem Cell phenotype. A fundamental issue for culture and expansion of mesenchymal stem cells and their use in regenerative medicine approaches is their tendency to rapidly lose their stem cell characteristics upon culture. In order to test if the treatment with soluble Collagen VI maintains hMSC phenotype, we analyzed the expression of the characteristic stem cell markers on the cell surface of hMSC, CD105 and CD90 by fluorescence activated cell sorting (FACS) (FIG. 9A). We observed that Col VI treated cell populations were similar to untreated cell populations with both retaining high expression of CD90 and CD105 after 2 days of Col VI treatment. It is to be noted that at a single cell level, 99% of hMSC were double positive for CD90 and CD105 both before and after ColVI treatment. Furthermore, ColVI treatment did not alter the expression of the chondrogenic transcription factor Sox9 and the cartilage surface marker CD44 in hMSC as assayed by FACS before and after 2 days of ColVI treatment (FIG. 9B). Even at gene expression level, expression of chondrogenic markers Sox9, Acan and Col2a1 was unchanged upon Col. VI treatment (FIG. 9C) suggesting that ColVI treatment did not skew the hMSC towards a chondrogenic lineage.

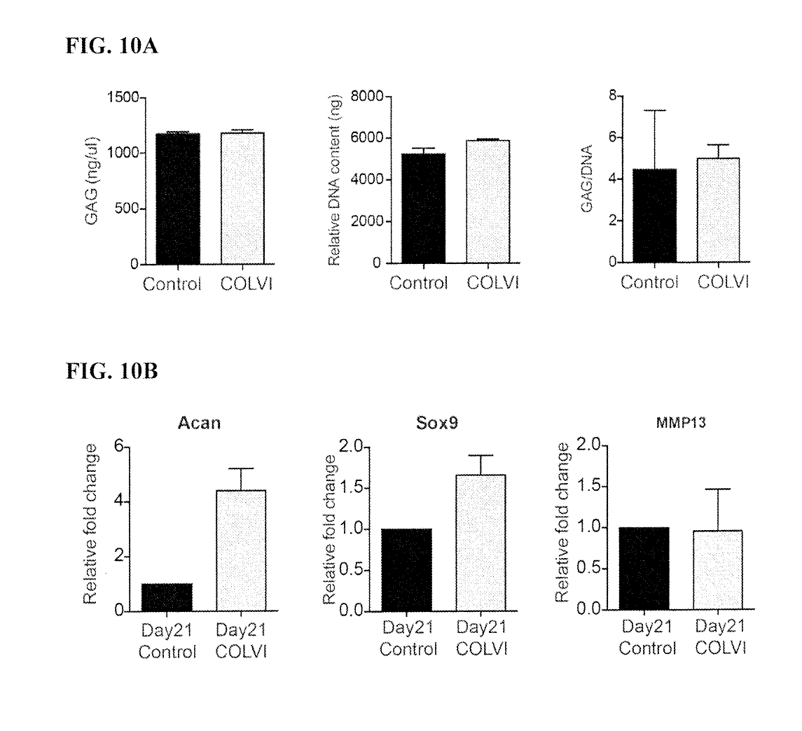

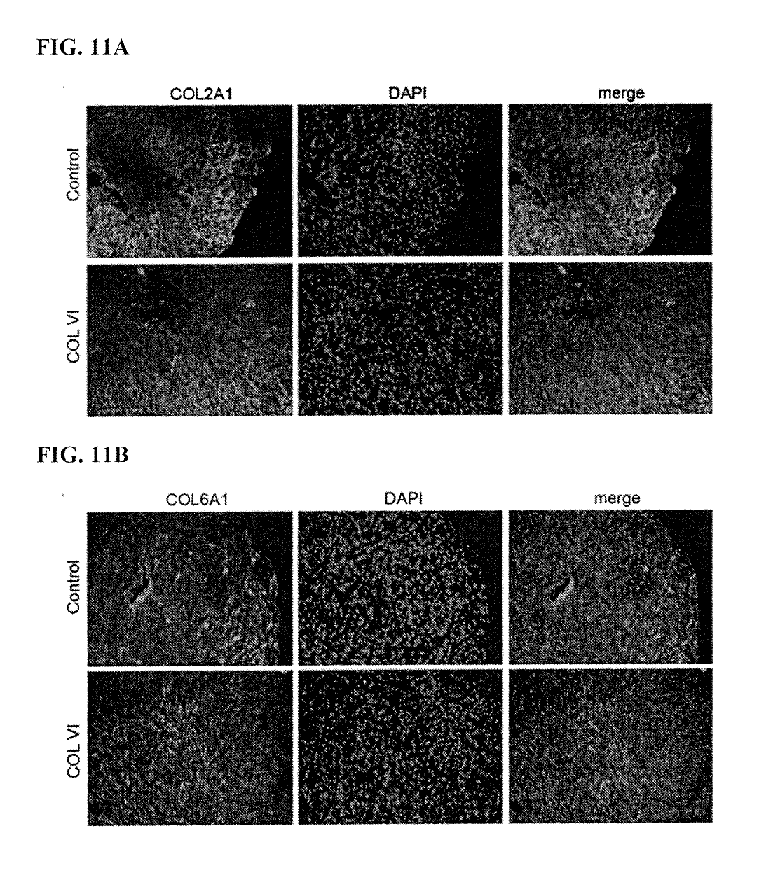

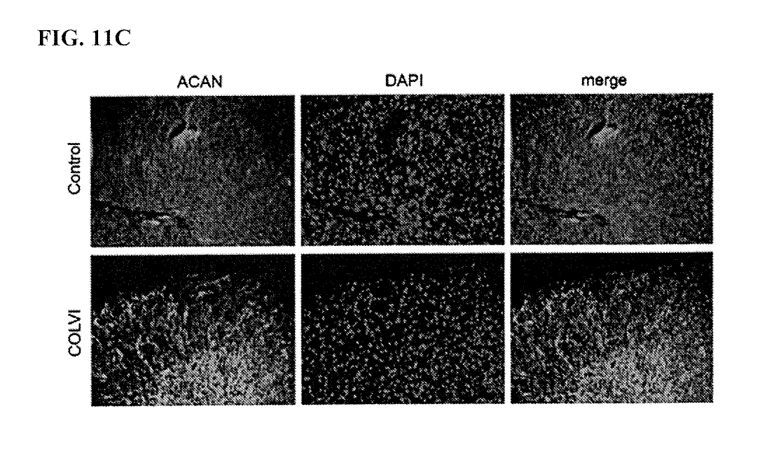

Col VI treatment retains the chondrogenic differentiation potential of hMSC. To assess if the COL VI treatment retains the potential of hMSC to differentiate into mature chondrocytes and engineer cartilage, we performed a three-dimensional pellet culture of chondrocytes after control or Col VI treatment (2.5 μgimp for 2 days. hMSC were treated with control or Col VI in monolayer culture, then centrifuged to allow pellet formation (500,000 cells per pellet) and cultured in TGF-beta enriched media for 21 days. At the end of the chondrogenic differentiation, pellets were analyzed for chondrogenic gene and protein expression expression as well as biochemical properties. We first observed that the DNA content in pellets of cells previously exposed to Col VI was not significantly higher than untreated control at the end of differentiation demonstrating that the proliferative effect is reversible and not sustained over time in the absence of the soluble Col VI (FIG. 10A). Furthermore, the Glycosaminoglycans (GAG) content quantification showed full maturation of cell pellets in both ColVI treatment and untreated control cells (FIG. 10A) suggesting that the chondrogenic potential of hMSC is retained upon Col VI treatment. Additionally, we observed that the gene expression of chondrogenic genes like Sox9 and Aggrecan was increased upon differentiation in Col VI treated cells. As observed previously, no increase in chondrogenic markers was observed in Col VI-treated and undifferentiated hMSC, however the prior Col VI-treatment appears beneficial to the chondrogenic differentiation of mesenchymal stem cells (FIG. 10B). MMP13 gene expression was unchanged by Col VI treatment (FIG. 10B). Histological evaluation and immunostaining of the cell pellets after 21 days of three dimensional pellet culture showed similar level of expression for cartilage specific Col2a1 and Aggrecan, again confirming that Col VI treated hMSC attained a mature chondrogenic phenotype similar to the control cells. Staining for endogenous ColVI was similar for the control and Col VI treated hMSC showing the absence of any feedback regulation of the soluble Col VI on the endogenous Col6a1 locus (FIGS. 11A-11C).

Discussion.

Although the utility of MSC in cartilage tissue engineering has been in focus for many years, recent studies are highlighting the benefits of MSC as a ‘secretory’ reserve of anti-inflammatory factors in addition to their ability to differentiate into cartilage (barry and murphy 2013 Nat rev rheum). The characteristics that make MSC an attractive candidate for knee Osteoarthritis are their safety and lack of immune rejection in the secluded knee joint. Indeed, a recently reported phase II clinical trial in a small patient cohort (9 patients) reported no adverse effects associated with adipose-derived stem cells in the secluded knee joint and showed improved pain scores as well as cartilage regeneration attributed to both anti-inflammatory effects and the ability of the stem cells to differentiate into cartilage (Ref-Stem cells). Scarcity of patient-derived MSC and a lack of effective methods for their expansion is the major barrier to the clinical translation of this attractive approach. Endogenous, cartilage-specific signals will be ideal for modulating the MSC populations in the knee joint as adverse effects in the native tissue will be minimal. Therefore, identification of Col VI as a soluble biologic for efficient expansion of MSC can be applied to both cartilage tissue engineering and modulation of inflammation in OA.

In the present study, we have identified the ability of soluble Col VI to expand bone-marrow derived human MSC in a 2D monolayer culture while maintaining a high expression of the MSC cell-surface markers like CD90 and CD105. These observations suggest that Col VI treatment would potentially be beneficial for other non-cartilage diseases as well as the treatment maintains the stemness of the mesenchymal populations and does not cause differentiation towards a chondrogenic lineage. The next question to address was whether the potential of MSC to engineer cartilage was affected by the expansion in the presence of soluble Col VI. Both control and Col VI-treated MSC showed robust cartilage tissue generation when seeded in equal numbers and cultured in 3D pellets for 3 weeks. Expression of chondrogenic markers like Age, Sox9, Col2a1 and Col6a1 was maintained or enhanced after 3 weeks of culture in both control and Col VI-treated MSC. Similarly, all the cartilage pellets showed robust Col II and AGC protein expression as well as high expression for Sox9 and Col VI while Col I and Col X expression remained minimal. In addition to the enhanced expression of chondrogenic factors in 3D culture, there was an equivalent accumulation of total GAG with or withour expansion in the presence of ColVI for 2 days. Therefore, the Col VI—expanded MSC effectively engineer cartilage tissue, comparable to MSC that have not been treated with Col VI.

In this study, we have described a useful and effective method for MSC expansion utilizing a cartilage-specific factor, Col VI. Identification of such endogenous cartilage-specific factors that can support and enhance stem cell function is likely key to stronger and effective cartilage regeneration strategies and OA modulation and treatment.

1. An in vitro mesenchymal stem cell culture comprising a plurality of mesenchymal stem cells and an amount of a protein selected from chordin-like protein 1 (CHRDL1) and collagen VI effective to increase in vitro growth, expansion or survival of said plurality of mesenchymal stem cells relative to the absence of said protein.

2. The mesenchymal stem cell culture of claim 1 comprising a liquid culture media.

3. The mesenchymal stem cell culture of claim 2 wherein said protein is soluble in said liquid culture media.

4. A method of increasing growth, expansion or survival of a plurality of mesenchymal stem cells, the method comprising contacting said plurality of mesenchymal stem cells with an effective amount of a protein selected from chordin-like protein 1 (CHRDL1) and collagen VI thereby increasing growth, expansion or survival of a plurality of mesenchymal stem cells.

5. A method of administering a plurality of mesenchymal stem cells to a subject in need thereof, the method comprising
a. contacting in vitro said plurality of mesenchymal stem cells with an effective amount of a protein selected from chordin-like protein 1 (CHRDL1) and collagen VI thereby increasing growth, expansion or survival of said plurality of mesenchymal stem cells to form a treated plurality of mesenchymal stem cells; and
b. administering said treated plurality of mesenchymal stem cells to said subject.

6. The method of claim 5, wherein said subject is a in need of a transplantation procedure.

7. The method of claim 6 further comprising transplanting additional biological tissue to said subject.

8. The method of claim 5 wherein said subject is in need of cartilage growth such as cartilage repair.

9. The method of claim 8 wherein said isolated mesenchymal stem cells differentiate to cartilage cells after said administering.

10. The method of claim 5 further comprising administering a protein selected from chordin-like protein 1 (CHRDL1) and collagen VI.

11. A method of increasing cartilage growth in a subject in need thereof, the method comprising administering an effective amount of a protein selected from chordin-like protein 1 (CHRDL1) and collagen VI.

12. The method of claim 11 further comprising administering a plurality of mesenchymal stem cells.

13. The method of claim 11 or 12 wherein said administering is through injection.

14. The method of claim 13 wherein said injection is provided at or near a cartilage-deficient site.
